(19) United States
(12) Patent Application Publication
(10) Pub. No.: US 2010/0028270 A1
(43) Pub. Date: Feb. 4, 2010

(54) URETHANE COMPOUND COMPRISING AN INCORPORATED POLYETHER GROUP-CONTAINING SILICONE DERIVATIVE AND A NITROGEN HETEROCYCLE

(75) Inventor: Son Nguyen-Kim, Hemsbach (DE)

Correspondence Address: CONNOLLY BOVE LODGE & HUTZ LLP, 1875 EYE STREET, N.W., SUITE 1100, WASHINGTON, DC, 20006, US

(73) Assignee: BASF AKTIENGESELLSCHAFT, Ludwigshafen (DE)

(21) Appl. No.: 11/722,189

(22) PCT Filed: Dec. 22, 2005
(86) PCT No.: PCT/EP2005/013927

§ 371 Date: Jun. 20, 2007

(30) Foreign Application Priority Data
Dec. 23, 2004 (DE) 10 2004 062 201.9

(51) Int. Cl. A61K 31/787; C08G 77/06; A61K 8/06; A61Q 5/02; A61Q 5/12; A61Q 5/06; A61Q 19/10; A61Q 19/00

(52) U.S. Cl. 424/47; 528/27; 510/158; 424/78.03; 424/70.12

(57) ABSTRACT

The present invention relates to a urethane compound which comprises, in incorporated form, at least one polyether group-containing silicone derivative, at least one nitrogen heterocycle and at least one polyisocyanate, to cosmetic and pharmaceutical compositions which comprise such a urethane compound, and to the use of such a urethane compound.

The present invention relates to a urethane compound which comprises at least one polyether group-containing silicone derivative, at least one nitrogen heterocycle and at least one polyisocyanate in incorporated form, to cosmetic and pharmaceutical compositions which comprise such a urethane compound, and to the use of such a urethane compound.

Cosmetically and pharmaceutically acceptable water-soluble polymers are used widely. Thus, they serve, for example in soaps, creams and lotions, as formulating agents, e.g. as thickener, foam stabilizer, water absorbent, or else to alleviate the irritative effect of other ingredients or to improve the dermal application of active ingredients. Their task in hair cosmetics consists in influencing the properties of the hair. Thus, film-forming polymers are used for hair cosmetics, for example as setting agents, and/or they serve as conditioners for improving the dry and wet combability, the feel to the touch, the shine and the appearance, and impart antistatic properties to the hair. In pharmacy, film-forming polymers serve, for example, as coatings or binders for solid drug forms.

The provision of products with a complex profile of properties often presents difficulties. There is thus a need for cosmetic compositions which comprise polymers which impart a pleasant feel to the hair and to the skin and at the same time have a good conditioning effect and/or setting effect. It is known that the sensory properties of the skin and/or of the hair can be improved through the use of silicone-containing polymers. In order, in addition, to achieve good setting and/or shaping of the hair, it is often necessary to formulate these silicone-containing polymers in combination with other hair polymers, e.g. acid group-containing polymers. There is thus a need for silicone-containing polymers for cosmetic and pharmaceutical compositions which on the one hand themselves have good application properties and on the other hand exhibit good compatibility with other formulation components.

WO 94/13724 describes the use of cationic polyurethanes and polyureas of
(a) at least one diisocyanate, which can already be reacted beforehand with one or more compounds which comprise two or more active hydrogen atoms per molecule, and
(b) at least one diol comprising one or more tertiary, quaternary or protonated tertiary amine nitrogen atoms, primary or secondary amino alcohol, primary or secondary diamine or primary or secondary triamine
   with a glass transition temperature of at least 25° C. and an amine number of from 50 to 200, based on the nonquaternized or protonated compounds, or other salts of these polyurethanes and polyureas as auxiliaries in cosmetic and pharmaceutical preparations.

WO 01/16200 describes a cosmetic composition comprising water-soluble or water-dispersible polyurethanes of an oligomer or polymer of
A) at least one diisocyanate,
B) at least one compound with at least two groups which are reactive toward isocyanate groups and which is chosen from
   B1) aliphatic and cycloaliphatic polyols, polyamines and/or amino alcohols,
   B2) polyetheroles and/or diaminopolyethers,
   B3) polysiloxanes with at least two active hydrogen atoms per molecule,
   B4) polyesterpolyoles,
   and mixtures thereof, and
C) if appropriate at least one dicarboxylic acid and/or hydroxycarboxylic acid,
   where the oligomer comprises, per molecule, at least two urethane and/or urea groups and additionally at least two further functional groups which are chosen from hydroxyl groups, and primary and/or secondary amino groups.

EP-A-938 889 describes a cosmetic composition comprising at least one water-soluble or water-dispersible polyurethane of
a) at least one polymer with two active hydrogen atoms per molecule which is chosen from polytetrahydrofurans, polysiloxanes and mixtures thereof,
b) at least one polyesterdiol,
c) at least one compound with a molecular weight in the range from 56 to 300 which comprises two active hydrogen atoms per molecule,
d) at least one compound which has two active hydrogen atoms and at least one anionogenic and/or anionic group per molecule,
e) at least one diisocyanate,
   or the salts thereof, where the polyurethane comprises no unit originating from a primary or secondary amine which has an ionogenic and/or ionic group.

WO 99/58100 describes a cosmetic composition comprising at least one crosslinked, water-soluble or water-dispersible polyurethane of at least one polyurethane prepolymer with terminal isocyanate groups and at least one polymer with groups which are reactive toward isocyanate groups, where at least one of the components comprises a siloxane group.

WO 01/85821 describes polyurethanes based on at least one polyether with two active hydrogen atoms per molecule and their use for modifying rheological properties.

WO 04/055088 describes allyl-group-containing polyether urethanes, polymers which comprise these in copolymerized form, and cosmetic or pharmaceutical compositions based on these polymers.

EP-A-957 119 describes crosslinked, water-soluble or water-dispersible polyurethanes of
A) at least one water-soluble or water-dispersible polyurethane preparation with terminal isocyanate groups of
   a) at least one compound with a molecular weight in the range from 56 to 300 which comprises two active hydrogen atoms per molecule,
   b) at least one polymer with two active hydrogen atoms per molecule,
   c) at least one compound which has two active hydrogen atoms and at least one ionogenic and/or ionic group per molecule,
   d) at least one diisocyanate,
B) at least one polymer with groups which are reactive toward isocyanate groups and which are chosen from hydroxyl groups, primary and secondary amino groups and/or carboxyl groups,
   or the salts thereof, and the use of these polyurethanes as auxiliaries in cosmetics.

WO 03/085019 describes crosslinked polyurethanes based on polytetrahydrofuran and its use in cosmetic and pharmaceutical compositions.

WO 99/04750 describes the use of water-soluble or water-dispersible polymers which are obtainable by free-radical polymerization of ethylenically unsaturated monomers in the presence of polyalkylene oxide-containing silicone derivatives for cosmetic formulations, and WO 01/13884 describes the use of these polymers in pharmaceutical preparations.

The unpublished German patent application P 102004036146.0 discloses a crosslinked polyurethane which comprises, in incorporated form,
A) at least one polytetrahydrofuran with two terminal hydroxyl groups per molecule and a number-average molecular weight in the range from 650 to 2000,
B) at least one compound which comprises more than two active hydrogen atoms per molecule,
C) at least one compound which comprises at least two active hydrogen atoms and at least one anionogenic and/or anionic group per molecule,
D) a polyisocyanate mixture which comprises isophorone diisocyanate and hexamethylene diisocyanate, and
E) at least one compound having at least two active hydrogen atoms and a molecular weight of from 60 to 5000 which is different from A) to D).

In addition, this polyurethane can comprise a polysiloxane with at least two active hydrogen atoms in incorporated form.

It is an object of the present invention to provide novel siloxane group-containing compounds which are suitable for the preparation of cosmetic and/or pharmaceutical compositions. Besides having an overall good application profile for this area of use, these compounds should primarily have good compatibility with other hair polymers, in particular those containing acid groups.

Surprisingly, it has now been found that this object is achieved by urethane compounds which comprise at least one polyether group-containing silicone derivative and at least one nitrogen heterocycle in incorporated form. The invention therefore provides a urethane compound A) which comprises, in incorporated form,
a) at least one polyether group-containing silicone derivative which comprises at least two groups which are reactive toward isocyanate groups,
b) at least one heterocycle with at least one ring nitrogen atom and at least one group which is reactive toward isocyanate groups and
c) at least one polyisocyanate.

A suitable embodiment of the urethane compounds according to the invention covers those which additionally comprise, in incorporated form, as component d) at least one compound which is different from components a) and b) and which comprises at least two groups which are reactive toward isocyanate groups.

Within the scope of the present invention, the expression alkyl comprises straight-chain and branched alkyl groups. Suitable short-chain alkyl groups are, for example, straight-chain or branched C₁-C₇-alkyl groups, preferably C₁-C₆-alkyl groups and particularly preferably C₁-C₄-alkyl groups. These include, in particular, methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, etc. Suitable longer-chain C₈-C₃₀-alkyl and C₈-C₃₀-alkenyl groups are straight-chain and branched alkyl and alkenyl groups. Preferably, these are predominantly linear alkyl radicals, as also arise in natural or synthetic fatty acids and fatty alcohols, and oxoalcohols which may if appropriate be additionally mono-, di- or polyunsaturated. These include, for example, n-hexyl(ene), n-heptyl(ene), n-octyl(ene), n-nonyl(ene), n-decyl(ene), n-undecyl(ene), n-dodecyl(ene), n-tridecyl(ene), n-tetradecyl(ene), n-pentadecyl(ene), n-hexadecyl(ene), n-heptadecyl(ene), n-octadecyl(ene), n-nonadecyl(ene) etc.

Cycloalkyl is preferably C₅-C₈-cycloalkyl, such as cyclopentyl, cyclohexyl, cycloheptyl or cyclooctyl.

Aryl comprises unsubstituted and substituted aryl groups and is preferably phenyl, tolyl, xylyl, mesityl, naphthyl, fluorenyl, anthracenyl, phenanthrenyl, naphthacenyl and in particular phenyl, tolyl, xylyl or mesityl.

Within the scope of the present invention, water-soluble monomers and polymers are understood as meaning monomers and polymers which dissolve in water in an amount of at least 1 g/l at 20° C. Hydrophilic monomers are water-soluble or at least water-dispersible. Water-dispersible polymers are understood as meaning polymers which disintegrate into dispersible particles under the application of shear forces, for example by stirring. The urethane compounds according to the invention are preferably water-soluble or water-dispersible. The water-dispersible urethane compounds according to the invention are generally suitable for producing microdispersions.

### Component a)

Component a) is preferably a polysiloxane with a number-average molecular weight in the range from about 300 to 50 000, particularly preferably 400 to 30 000.

Preferred compounds of component a) are polysiloxanes of the general formula I.1

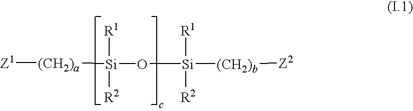

in which
a and b, independently of one another, are 1 to 8,
c is 2 to 1000,
R¹ and R², independently of one another, are alkyl, cycloalkyl or aryl,
Z¹ and Z², independently of one another, are radicals of the formula II

—(OCH₂CH₂)ᵤ(OCH(CH₃)CH₂)ᵥ(O(CH₂)₄)_{w}—X¹—H (II)

where
in formula II the order of the alkylene oxide units is arbitrary,
u, v and w, independently of one another, are an integer from 0 to 500, where the sum of u, v and w>0,
X¹ is O or NR³, in which R³ is hydrogen, alkyl, cycloalkyl or aryl.

Preferably, in the formula I.1 the sum of u, v and w is chosen such that the molecular weight of the polysiloxanes a) is in a range from about 300 to 30 000.

Preferably, the total number of alkylene oxide units in the polysiloxanes a), i.e. the sum of u, v and w, is in a range from about 3 to 200, preferably 5 to 180.

Preferably, in the compounds of the formula I, the radicals R¹ and R², independently of one another, are chosen from methyl, ethyl, cyclohexyl, phenyl and benzyl. Particularly preferably, R¹ and R² are both methyl.

One example of suitable compounds of the formula I.1 are the bis(polyethylene glycol) dimethicones of the general formula I.1a

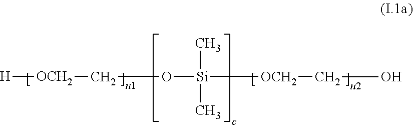

in which
c is an integer from 3 to 500, preferably 5 to 250, and
u1 and u2, independently of one another, are 2 to 500, in particular 3 to 250, specifically 5 to 100.

Preferably, the compounds of component a) are also chosen from polysiloxanes of the general formula I.2

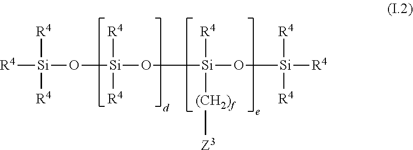

in which
the order of the siloxane units is arbitrary, the radicals R⁴, independently of one another, are in each case alkyl, cycloalkyl or aryl,
d is an integer from 2 to 1000,
e is an integer from 2 to 100,
f is an integer from 2 to 8, and
Z³ is a radical of the formula II as defined above,
   and mixtures thereof.

One example of suitable compounds of the formula I.2 covers the ethoxylated and/or propoxylated polydimethylsiloxanes of the general formula I.2a

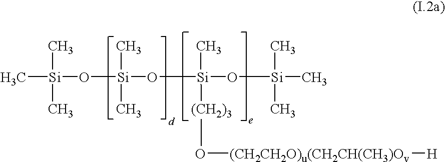

in which
the order of the siloxane units is arbitrary,
d is an integer from 2 to 1000, preferably 3 to 500, in particular 5 to 100,
e is an integer from 2 to 100, preferably 3 to 50, in particular 4 to 20, and
u and v, independently of one another, are an integer from 0 to 500, preferably 0 to 250, where the sum of u and v is ≧1, preferably ≧5, in particular ≧10.

Suitable compounds of the formula I.1 are available under the name Wacker-Belsil® DMC 6031 and Pluriolφ ST 4005 (BASF Aktiengesellschaft).

Suitable compounds of component a) are quite generally siloxane derivatives which have at least two polyether chains with terminal OH groups. These include the compounds known under the INCI names dimethicone copolyols or silicone surfactants, as are obtainable under the brand names Abil®, Degussa-Goldschmidt, Alkasil® (Rhone Poulenc), Silicone Polyol Copolyol® (Genesee Polymers Corporation), Belsil® (Wacker), Silwet® (OSI), Dow Corning (Dow Corning) or Tecopren (Degussa-Goldschmidt).

### Component b)

The heterocycles used according to the invention as component b) have at least one ring nitrogen atom and additionally at least one group which is reactive toward isocyanate groups. This group which is reactive toward NCO groups may be at least one further ring nitrogen atom to which a hydrogen atom is bonded. In addition, the group which is reactive toward NCO groups may be a group chosen from hydroxyl, mercapto, primary and secondary amino groups which is bonded to a ring atom via a chemical bond or a bridging group. Suitable bridging groups are, for example, C₁-C₁₀-alkylene groups, such as —CH₂—, —CH₂CH₂— etc.

The heterocycles used as component b) preferably have no vinyl group or alkyl group bonded to a ring atom. That means component b) is preferably not N-vinylimidazole or a derivative thereof.

Preferably, at least one of the ring nitrogen atoms is sp²-hybridized.

Preferably, component b) is chosen from nitrogen-containing heterocycles from the group of purines, pyrazoles, imidazoles, triazoles, tetrazoles, piperazines, imidazolines, imidazolidines, pyrazolines, pyrazolidines and mixtures thereof.

The abovementioned nitrogen-containing heterocycles may be unsubstituted or substituted. Depending on the number of ring carbon atoms, substituted heterocycles preferably have 1, 2 or 3 substituents. These substituents are preferably chosen from alkyl, cycloalkyl, heterocycloalkyl, aryl, hetaryl, hydroxy, thiol, polyalkylene oxide, polyalkyleneimine, alkoxy, cycloalkoxy, aryloxy, COOH, carboxylate, SO₃H, sulfonate, alkoxycarbonyl, acyl and nitro.

Component b) is also preferably chosen from nitrogen-containing heterocycles from the group of pyridines, pyridazines, pyrimidines, pyrazines, 1,3,5-triazines, 1,2,4-triazines, 1,2,3-triazines, tetrazines, pyrroles, quinolines, isoquinolines, cinnolines, quinazolines, quinoxalines, phenazines, acridines, indoles, isoindoles, carbazoles, pyrrolines and pyrrolidines. These heterocycles then have at least one group which is reactive toward isocyanate groups as substituents. In addition, these heterocycles can have at least one further substituent, which is preferably chosen from alkyl, cycloalkyl, heterocycloalkyl, aryl, hetaryl, alkoxy, cycloalkoxy, aryloxy, COOH, carboxylate, SO₃H, sulfonate, alkoxycarbonyl, acyl and nitro. Suitable heterocycles b) are, for example, 3-hydroxypiperidine, 4-hydroxypiperidine, 2-hydroxypyridine, 3-hydroxypyridine, 4-hydroxypyridine, 2-aminopyridine, 3-aminopyridine, 4-aminopyridine, 2-aminopyrimidine, 4-aminopyrimidine, aminopyridinol, 2-picolylamine, 3-picolylamine, 4-picolylamine, 3-amino-5-methylpyrazole, 2-amino-4-methylpyrimidine and mixtures thereof.

Preferably, component b) comprises imidazole or a derivative thereof. Preferred compounds b) are imidazole and imidazole derivatives of the general formula (II),

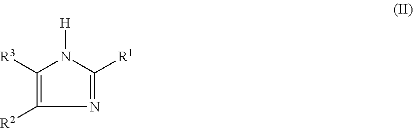

in which R¹, R² and R³, independently of one another, are chosen from hydrogen, C₁-C₄-alkyl, specifically methyl, ethyl, n-propyl, isopropyl, n-butyl and tert-butyl, C₅-C₆-cycloalkyl, specifically cyclohexyl, and C₆-C₁₀-aryl, specifically phenyl.

Examples of compounds of the general formula (II) are given in Table 1 below:

TABLE 1

| R¹ | R² | R³ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

A further preferred imidazole derivative is histidine.

Particularly preferably, component b) comprises imidazole or consists of it.

### Component c)

Suitable polyisocyanates c) are chosen from compounds with 2 to 5 isocyanate groups, isocyanate prepolymers with an average number of from 2 to 5 isocyanate groups, and mixtures thereof. These include, for example, aliphatic, cycloaliphatic and aromatic di-, tri- and polyisocyanates. Suitable diisocyanates c) are, for example, tetramethylene diisocyanate, hexamethylene diisocyanate, 2,3,3-trimethylhexamethylene diisocyanate, 1,4-cyclohexylene diisocyanate, isophorone diisocyanate, 1,4-phenylene diisocyanate, 2,4- and 2,6-tolylene diisocyanate and isomer mixtures thereof (e.g. 80% 2,4- and 20% 2,6-isomer), 1,5-naphthylene diisocyanate, 2,4- and 4,4′-diphenylmethane diisocyanate. A suitable triisocyanate is, for example, triphenylmethane 4,4′,4″-triisocyanate. Also suitable are isocyanate prepolymers and polyisocyanates, which are obtainable by addition of the abovementioned isocyanates onto polyfunctional hydroxyl- or amine group-containing compounds. Also suitable are polyisocyanates which arise through biuret, allophanate or isocyanurate formation.

Preferably, component c) comprises at least one diisocyanate with two isocyanate groups of varying reactivity. Particularly preferably, component C) comprises exclusively isophorone diisocyanate and its biurets, allophanates and/or isocyanurates. In particular, component C) consists only of isophorone diisocyanate.

### Component d)

In a further embodiment, the urethane compounds according to the invention comprise, in incorporated form, at least one compound d) which is preferably chosen from
d1) compounds with a molecular weight in the range from 56 to 280 g/mol which comprise two groups which are reactive toward isocyanate groups per molecule,
d2) polymers with a number-average molecular weight of more than 280 which have two groups which are reactive toward isocyanate groups per molecule, and mixtures thereof.

Suitable compounds d1) are, for example, diols, diamines, amino alcohols and mixtures thereof.

As component d1), preference is given to using diols whose molecular weight is in a range from about 62 to 286 g/mol. These include, for example, diols with 2 to 18 carbon atoms, preferably 2 to 10 carbon atoms, such as 1,2-ethanediol, 1,3-propanediol, 1,4-butanediol, 1,6-hexanediol, 1,5-pentanediol, 1,10-decanediol, 2-methyl-1,3-propanediol, 2,2-dimethyl-1,3-propanediol, di-, tri-, tetra-, penta- and hexaethylene glycol, neopentyl glycol, cyclohexanedimethylol and mixtures thereof. Particular preference is given to neopentyl glycol.

Preferred amino alcohols d1) are, for example, 2-aminoethanol, 2-(N-methylamino)-ethanol, 3-aminopropanol, 4-aminobutanol, 1-ethylaminobutan-2-ol, 2-amino-2-methyl-1-propanol, 4-methyl-4-aminopentan-2-ol etc.

Preferred diamines d1) are, for example, ethylenediamine, propylenediamine, 1,4-diaminobutane, 1,5-diaminopentane and 1,6-diaminohexane.

The compounds specified as component d1) can be used individually or in mixtures. Particular preference is given to using 1,2-ethanediol, 1,4-butanediol, 1,6-hexanediol, neopentyl glycol, diethylene glycol, cyclohexanedimethylol and mixtures thereof.

Component d2) is preferably a polymer with a number-average molecular weight in the range from about 300 to 5000, particularly preferably about 400 to 4000, in particular 500 to 3000. Polymers d2) which can be used are, for example, polyesterdiols, polyetherols and mixtures thereof.

Preferred polyetherols d2) are polyalkylene glycols, e.g. polyethylene glycols, polypropylene glycols, polytetrahydrofurans, etc., copolymers of ethylene oxide and propylene oxide or block copolymers of ethylene oxide, propylene oxide and/or butylene oxide which comprise the copolymerized alkylene oxide units in random distribution or in the form of blocks. Suitable polytetrahydrofurans d2) may be produced by cationic polymerization of tetrahydrofuran in the presence of acidic catalysts, such as, for example, sulfuric acid or fluorosulfuric acid. Such production methods are known to the person skilled in the art. Suitable compounds d2) are also α,ω-diaminopolyethers, which can be produced by amination of polyalkylene oxides with ammonia. As component E2), preference is given to using polyesterdiols and mixtures which comprise these.

Suitable polyesterdiols d2) preferably have a number-average molecular weight in the range from about 400 to 5000, preferably 500 to 3000, in particular 600 to 2000. Suitable polyesterdiols d2) are all those which are customarily used for producing polyurethanes, in particular those based on aromatic dicarboxylic acids, such as terephthalic acid, isophthalic acid, phthalic acid, Na or K sulfoisophthalic acid etc., aliphatic dicarboxylic acids, such as adipic acid or succinic acid etc., and cycloaliphatic dicarboxylic acids, such as 1,2-, 1,3- or 1,4-cyclohexanedicarboxylic acid. Suitable diols are, in particular, aliphatic diols, such as ethylene glycol, propylene glycol, 1,6-hexanediol, neopentyl glycol, diethylene glycol, polyethylene glycols, polypropylene glycols, 1,4-dimethylolcyclohexane.

Preference is given to polyesterdiols d2) based on aromatic and aliphatic dicarboxylic acids and aliphatic diols, in particular those in which the aromatic dicarboxylic acid constitutes 10 to 95 mol %, in particular 40 to 90 mol %, of the total dicarboxylic acid fraction (remainder aliphatic dicarboxylic acids).

Particularly preferred polyesterdiols d2) are the reaction products of phthalic acid/diethylene glycol, isophthalic acid/1,4-butanediol, isophthalic acid/adipic acid/-1,6-hexanediol, 5-NaSO₃-isophthalic acid/phthalic acid/adipic acid/1,6-hexanediol, adipic acid/ethylene glycol, isophthalic acid/adipic acid/neopentyl glycol, isophthalic acid/adipic acid/neopentyl glycol/diethylene glycol/dimethylolcyclohexane and 5-NaSO₃-isophthalic acid/isophthalic acid/adipic acid/neopentyl glycol/diethylene glycol/dimethylolcyclohexane, isophthalic acid/adipic acid, neopentyl glycol/dimethylol-cyclohexane.

Preferred components d2) are also polyesterdiols based on linear or branched C₈-C₃₀-di- or polycarboxylic acids and C₈-C₃₀-hydroxycarboxylic acids. Preferred carboxylic acids and hydroxycarboxylic acids are, for example, azelaic acid, dodecanedioic acid, suberic acid, pimelic acid, sebacic acid, tetradecanedioic acid, citric acid, ricinoleic acid, hydroxystearic acid and mixtures thereof. Diol components used for producing these polyesterdiols are preferably 1,4-butanediol, 1,5-pentanediol, 1,6-hexanediol, neopentyl glycol, 1,4-dimethylolcyclohexane, diethylene glycol and mixtures thereof.

The urethane compounds according to the invention are preferably produced by a two-stage method. The components are reacted here preferably such that the resulting urethane compounds have at least two terminal heterocycles b). With these heterocycles, preferably at least one of the ring nitrogen atoms is not bonded in the form of a urethane and/or urea group.

The invention thus also provides a method of producing a urethane compound which comprises, in incorporated form,
a) at least one polyether group-containing silicone derivative which comprises at least two groups which are reactive toward isocyanate groups,
b) at least one heterocycle with at least one ring nitrogen atom and at least one group which is reactive toward isocyanate groups
c) at least one polyisocyanate, and
d) if appropriate at least one compound which is different from components a) and b) and which comprises at least two groups which are reactive toward isocyanate groups,
   in which
i) in a first stage the compounds a) and the polyisocyanates c) and, if present, if appropriate at least some of the compounds d) are reacted to give an isocyanate group-containing prepolymer, and
ii) in a second stage the prepolymer present in i) is reacted with the compounds b) and, if present, the compounds d) which have not already been used in step i).

In the first stage i), an NCO group-containing prepolymer is prepared from the polyether group-containing silicone derivatives a) and the polyisocyanates c). If desired, some or all of the compounds of component d) can be used for producing the prepolymer in this stage. In this case, suitable choice of the amount of component c) ensures that an isocyanate group-containing prepolymer is nevertheless obtained. The ratio of NCO equivalents of component c) to equivalents of active hydrogen atoms of components a) and, if present, d) is preferably in a range from about 1:1 to 3:1, particularly preferably 1.01:1 to 2.5:1, in particular 1.05:1 to 2:1.

The reaction in stage i) is preferably carried out without solvents. However, it is also possible to carry out the reaction in stage i) in a suitable inert solvent or solvent mixture. Suitable solvents are aprotically polar solvents, e.g. tetrahydrofuran, ethyl acetate, N-methylpyrrolidone, dimethylformamide and preferably ketones, such as acetone and methyl ethyl ketone. Preferably, the reaction is carried out under an inert gas atmosphere, such as, for example, under nitrogen. Furthermore, the reaction is preferably carried out at ambient pressure or under increased pressure. The reaction temperature is preferably in a range from about 0 to 120° C., particularly preferably 5 to 90° C. If the components a) and, if present, d) comprise amine group-containing compounds, then the reaction temperature is preferably in a range from about 0 to 60° C., particularly preferably from 10 to 40° C. If amine group-containing components are used, the reaction can, if desired, also be carried out in a solvent or solvent mixture, which may have active hydrogen atoms. Besides those specified above, preference is then given to using cosmetically acceptable solvents, preferably alcohols, such as ethanol and isopropanol and mixtures of alcohols and water.

The NCO group-containing prepolymers obtained in step i) can, if desired, be subjected to isolation and/or purification by customary methods known to the person skilled in the art prior to further reaction in step ii). The production of the prepolymers and the production of the urethane compounds A) according to the invention therefrom preferably takes place without isolation of an intermediate product. The urethane compound according to the invention in step ii) is produced by reacting the prepolymers obtained in step i) with the heterocycles b) and, if appropriate, compounds of component d). Here, the ratio of NCO equivalents of the prepolymers to equivalents of active hydrogen atoms of components b) and, if present, d) is in a range from about 0.6:1 to 1.4:1, preferably 0.8:1 to 1.2:1, in particular 0.9:1 to 1.1:1.

The reaction in the second stage is also preferably carried out without solvents. However, it is also possible to carry out the reaction in stage ii) in one of the abovementioned solvents, preferably in a cosmetically acceptable solvent. If component b) is bonded via amine groups, and also if any compounds d) used have amine groups as NCO reactive groups, the reaction can be carried out in alcohols and alcohol/water mixtures. Preference is given to ethanol, isopropanol, mixtures thereof and mixtures of these alcohols with water. The reaction temperature in step ii) is then preferably in a range from about 0 to 60° C., particularly preferably 10 to 40° C. If the resulting urethane compounds still have free isocyanate groups, then these are finally deactivated by adding low molecular weight compounds with groups which are reactive toward NCO groups. These include amines, particularly preferably amino alcohols, alcohols, in particular ethanol, and water.

The two-stage production method according to the invention is particularly advantageously suitable for producing a urethane compound which comprises, in incorporated form, the above-described components a), b), c) and, if appropriate, d), where at least one of components a) and, if present, d) has at least one hydroxyl group-containing compound, in which
i) all of the hydroxyl group-containing compounds a) and d) and the polyisocyanates c) are reacted at a temperature in a range from 55 to 120° C. in the absence of a solvent or in an aprotically polar solvent to give an isocyanate group-containing prepolymer, and
ii) the prepolymer obtained in stage i) is reacted with the compounds b) and, if present, the compounds d) not already used in step i) at a temperature in a range from 0 to 60° C. in an alcohol or alcohol/water mixture as solvent.

The urethane compounds according to the invention preferably have 1 to 100, particularly preferably 2 to 40, in particular 3 to 25, heterocyclic groups derived from compounds b).

In a particular embodiment, the urethane compounds according to the invention additionally have amine groups which are not derived from heterocyclic compounds b) and which are not bonded in amidic form (e.g. as polyurethane or polyurea). Such urethane compounds are obtained, for example, if component a) comprises silicone polyalkylene oxide copolymers which also have amine group-containing side chains besides polyalkylene oxide side chains. These may, for example, be hydrocarbon side chains with internal (secondary and/or tertiary) or terminal (primary) amino groups. Such urethane compounds are also obtained if, in the production of the urethane compound or for deactivating any free isocyanate groups present, compounds are used which have a group which is reactive toward NCO groups and additionally at least one tertiary amino group. Preferably, these compounds are chosen from the following compounds

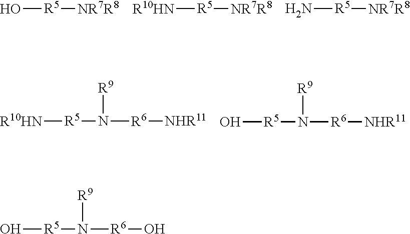

in which
R⁵ and R⁶, independently of one another, are C₂-C₈-alkylene,
R⁷, R⁸ and R⁹, independently of one another, are alkyl, cycloalkyl or aryl, and
R¹⁰ and R¹¹, independently of one another, are hydrogen, alkyl, cycloalkyl or aryl.

Preferably, these compounds are also chosen from amino acids of the general formula

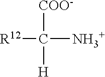

in which R¹² is a radical which has at least one group which is reactive toward isocyanate groups. These include serine, threonine, cysteine, tyrosine, lysine and arginine. Preference is given to the amino acids with basic groups in the side chain, lysine and arginine.

The number-average molecular weight of the urethane compounds according to the invention is preferably about 300 to 30 000, particularly preferably 500 to 25 000, in particular 1000 to 15 000.

The amine number of the urethane compounds is preferably in a range from 5 to 100, particularly preferably from 10 to 60.

Preferably, the urethane compounds comprise
40 to 98% by weight, particularly preferably 50 to 95% by weight, of at least one component a),
0.1 to 25% by weight, preferably 0.3 to 15% by weight, of at least one component b), 1.9 to 35% by weight, preferably 4.7 to 25% by weight, of at least one component c),
0 to 60% by weight, preferably 1 to 50% by weight, of at least one component d)
   in incorporated form.

Particular preference is given to urethane compounds which comprise, in incorporated form,
a) at least one polyether group-containing silicone derivative with a number-average molecular weight of from 1000 to 25 000 which comprises at least two hydroxyl groups,
b) imidazole and/or a derivative thereof,
c) isophorone diisocyanate, and
d) if appropriate at least one polyetherpolyol with a number-average molecular weight in the range from 200 to 5000.

To produce the urethane compounds A) according to the invention, the heterocycles b) and, if appropriate, further amine group-containing monomers are preferably used in an initially uncharged form. The urethane compounds according to the invention thus have cationogenic groups.

Preferably, the urethane compounds according to the invention comprise no copolymerized monomers with anionogenic and/or anionic groups.

The urethane compounds according to the invention have very good compatibility with a large number of acid group-containing polymers. They are therefore suitable in an advantageous manner for a partial or complete neutralization of such polymers, the resulting salts combining the advantageous properties of the urethane compounds according to the invention and of the acid group-containing polymers. If desired, the polyurethane compounds according to the invention can also be used in combination with further cosmetically and/or pharmaceutically compatible polymers with cationogenic and/or cationic groups. This polymer combination can then be used for a partial or complete neutralization of the anionogenic groups of acid group-containing polymers.

Of suitability for adjusting the pH of aqueous preparations of the urethane compounds according to the invention, alone or in combination with further polymers with cationogenic and/or anionogenic groups, are in principle all inorganic or organic acids and bases, in particular those which are water-soluble. Suitable acids are, for example, carboxylic acids, such as lactic acid, citric acid or tartaric acid, or mineral acids, such as phosphoric acid, sulfuric acid or hydrochloric acid. Suitable bases are, for example, alkali metal and alkaline earth metal hydroxides, ammonia, and primary, secondary and tertiary amines, such as triethylamine, and amino alcohols such as triethanolamine, methyldiethanolamine, dimethylethanolamine or 2-amino-2-methylpropanol. The urethane compounds A) described above are exceptionally suitable for producing cosmetic and pharmaceutical compositions. They serve here, for example, as polymeric conditioning agents in preparations for bodycare, in particular for hair-treatment compositions. They can be used and formulated universally into very diverse cosmetic and pharmaceutical preparations and are compatible with customary components. These include, in particular, further cosmetically or pharmaceutically acceptable polymers with acid groups, and cationic, amphoteric and neutral polymers. The salts of the urethane compound A) according to the invention, specifically the salts with acid group-containing polymers, are generally characterized by good solubility in water. Furthermore, the urethane compounds A) permit, in particular, the formulation of products in the form of sprays and are characterized by improved conditioner properties. They thus impart good sensory properties, such as smoothness, shine and suppleness, to the skin treated therewith and to the hair treated therewith. In addition, they can be washed out easily. In combination with acid group-containing polymers, they are additionally characterized by good film formation.

The urethane compounds according to the invention based on cationogenic heterocycles b) are advantageously suitable as oligomeric/polymeric neutralizing agent. They are specifically suitable for the partial or complete neutralization of further cosmetically or pharmaceutically acceptable polymers with anionogenic groups. Here, they can be used as the sole cationogenic neutralizing agent, in combination with at least one further cosmetically or pharmaceutically acceptable polymer with cationogenic groups and/or in combination with at least one of the abovementioned bases. The invention thus also provides cosmetic or pharmaceutical compositions which comprise at least one urethane compound A), as defined above and at least one acid group-containing polymer S).

The compositions according to the invention preferably comprise urethane compounds A) and polymers S) in a quantitative weight ratio of from about 0.1:100 to 20:100, particularly preferably from 0.5:100 to 15:100, in particular 1:100 to 10:100.

Suitable acid group-containing polymers S) are obtainable, for example, by free-radical polymerization of α,β-ethylenically unsaturated monomers. For this, use is made of monomers c.1) which comprise at least one free-radically polymerizable, α,β-ethylenically unsaturated double bond and at least one anionogenic and/or anionic group per molecule.

Suitable acid group-containing polymers S) are also acid group-containing polyurethanes.

The monomers c.1) are preferably chosen from monoethylenically unsaturated carboxylic acids, sulfonic acids, phosphonic acids and mixtures thereof.

The monomers c.1) include monoethylenically unsaturated mono- and dicarboxylic acids with 3 to 25, preferably 3 to 6, carbon atoms, which can also be used in the form of their salts or anhydrides. Examples thereof are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. The monomers c.1) further include the half-esters of monoethylenically unsaturated dicarboxylic acids with 4 to 10, preferably 4 to 6, carbon atoms, e.g. of maleic acid, such as monomethyl maleate. The monomers c.1) also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxypropylsulfonic acid, styrenesulfonic acid, 2-acrylamido-2-methylpropanesulfonic acid, vinylphosphonic acid and allylphosphonic acid. The monomers c. 1) also include the salts of the abovementioned acids, in particular the sodium, potassium and ammonium salts, and the salts with the abovementioned amines. The monomers c.1) can also be used as they are or as mixtures with one another. The weight fractions given all refer to the acid form.

Component c.1) is preferably chosen from acrylic acid, methacrylic acid and mixtures thereof.

In principle, suitable comonomers for the preparation of the polymers S) are all α,β-ethylenically unsaturated compounds which are copolymerizable with the monomers c.1).

In a particular embodiment, the polymers S) comprise at least one crosslinking monomer c.2) in copolymerized form. Suitable crosslinkers c.2) are compounds with two or more than two ethylenically unsaturated nonconjugated double bonds. Preference is given to using crosslinkers c.2) in an amount of from 0.01 to 3% by weight, particularly preferably 0.1 to 2% by weight, based on the total weight of the monomers used for the polymerization of the polymers S).

Suitable crosslinkers c.2) are, for example, acrylic esters, methacrylic esters, allylethers or vinylethers of at least dihydric alcohols. The OH groups of the parent alcohols here may be completely or partially etherified or esterified; however, the crosslinkers comprise at least two ethylenically unsaturated groups.

Further suitable crosslinkers c.2) are the vinyl esters or the esters of monohydric, unsaturated alcohols with ethylenically unsaturated C₃-C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. Further suitable crosslinkers c.2) are esters of unsaturated carboxylic acids with polyhydric alcohols. Suitable crosslinkers c.2) are also straight-chain or branched, linear or cyclic, aliphatic or aromatic hydrocarbons which have at least two double bonds, which in the case of aliphatic hydrocarbons must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000. Further suitable crosslinkers c.2) are the acrylamides, methacrylamides and N-allylamines of at least difunctional amines. Such amines are, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides of allylamine and unsaturated carboxylic acids, such as acrylic acid, methacrylic acid, itaconic acid, maleic acid, or at least dibasic carboxylic acids as have been described above. Also suitable are triallylamine and triallylmonoalkylammonium salts, e.g. triallylmethylammonium chloride or methylsulfate, as crosslinker c.2).

Also suitable are N-vinyl compounds of urea derivatives, at least difunctional amides, cyanurates or urethanes, for example of urea, ethyleneurea, propyleneurea or tartramide, e.g. N,N′-divinylethyleneurea or N,N′-divinylpropyleneurea. Further suitable crosslinkers c.2) are divinyidioxane, tetraallylsilane or tetravinylsilane. Particularly preferably used crosslinkers c.2) are, for example, methylenebisacrylamide, triallylamine and triallylalkylammonium salts, divinylimidazole, pentaerythritol triallyl ether, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin.

Furthermore, the polymers S) preferably comprise at least one monomer c.3) in copolymerized form which is chosen from compounds of the general formula III)

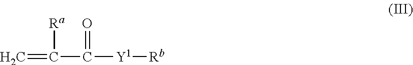

in which
R^{a} is hydrogen or C₁-C₈-alkyl,
Y¹ is O, NH or NR^{c}, and
R^{b} and R^{c}, independently of one another, are C₁-C₃₀-alkyl or C₅-C₈-cycloalkyl, where the alkyl groups may be interrupted by up to four nonadjacent heteroatoms or heteroatom-containing groups which are chosen from O, S and NH.

Preferably, R^{a} in formula I is hydrogen or C₁-C₄-alkyl, in particular hydrogen, methyl or ethyl.

Preferably, R^{b} in formula I is C₁-C₈-alkyl, preferably methyl, ethyl, n-butyl, isobutyl, tert-butyl or a group of the formula —CH₂—CH₂—NH—C(CH₃)₃.

If R^{c} is alkyl, then it is preferably C₁-C₄-alkyl, such as methyl, ethyl, n-propyl, n-butyl, isobutyl and tert-butyl.

Suitable monomers c.3) are methyl (meth)acrylate, methyl ethacrylate, ethyl (meth)acrylate, ethyl ethacrylate, tert-butyl (meth)acrylate, tert-butyl ethacrylate, n-octyl (meth)acrylate, ethylhexyl (meth)acrylate, stearyl (meth)acrylate, lauryl (meth)acrylate, etc. and mixtures thereof.

Suitable monomers c.3) are also acrylamide, methacrylamide, N-methyl(meth)acrylamide, N-ethyl(meth)acrylamide, N-propyl(meth)acrylamide, N-(n-butyl)(meth)acrylamide, N-(tert-butyl)(meth)acrylamide, N,N-dimethyl(meth)acrylamide, N,N-diethyl(meth)acrylamide, piperidinyl(meth)acrylamide and morpholinyl(meth)acrylamide, N-(n-octyl)(meth)acrylamide, N-ethylhexyl(meth)acrylamide, N-stearyl(meth)acrylamide, N-lauryl(meth)acrylamide, etc. and mixtures thereof.

Furthermore, the polymers C) preferably comprise at least one monomer c.4) in copolymerized form which is chosen from compounds of the general formula IV

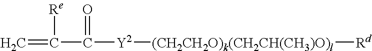

in which
the order of the alkylene oxide units is arbitrary,
k and l, independently of one another, are an integer from 0 to 1000, where the sum of k and l is at least 5,
R^{d} is hydrogen, C₁-C₃₀-alkyl or C₅-C₈-cycloalkyl,
R^{e} is hydrogen or C₁-C₈-alkyl,
Y² is O or NR^{f}, where R^{f} is hydrogen, C₁-C₃₀-alkyl or C₅-C₈-cycloalkyl.

Preferably, in formula IV is an integer from 1 to 500, in particular 3 to 250. Preferably, I is an integer from 0 to 100.

Preferably, R^{e} is hydrogen, methyl, ethyl, n-propyl, isopropyl, n-butyl, sec-butyl, tert-butyl, n-pentyl or n-hexyl, in particular hydrogen, methyl or ethyl.

Preferably, R^{d} in formula IV is hydrogen, methyl, ethyl, n-propyl, isopropyl, n-butyl, sec-butyl, n-pentyl, n-hexyl, octyl, 2-ethylhexyl, decyl, lauryl, palmityl or stearyl.

Preferably, Y² in formula IV is O or NH.

Preferably, the acid group-containing polymer S) comprises
acrylic acid and/or methacrylic acid,
at least one C₁-C₄-alkyl (meth)acrylate which is preferably chosen from methyl methacrylate, ethyl acrylate, ethyl methacrylate, tert-butyl acrylate and mixtures thereof,
at least one C₁-C₄-alkyl(meth)acrylamide, and
if appropriate at least one crosslinker
   in copolymerized form.

Furthermore, the acid group-containing polymer S) comprises
acrylic acid and/or methacrylic acid,
at least one monomer which is chosen from C₁-C₄-alkyl (meth)acrylates, C₁-C₄-alkyl(meth)acrylamides and mixtures thereof,
at least one polyether acrylate, and
if appropriate at least one crosslinker in copolymerized form.

Furthermore, the acid group-containing polymer S) preferably comprises
t-butyl acrylate and/or ethyl acrylate, and
acrylic acid and/or methacrylic acid
   in copolymerized form.

Furthermore, the acid group-containing polymer S) preferably comprises
at least one C₁-C₄-alkyl (meth)acrylate, preferably t-butyl acrylate,
vinylpyrrolidone and/or vinylcaprolactam, and
acrylic acid and/or methacrylic acid
   in copolymerized form.

Anionic polymers preferred as polymers S) are, for example, homopolymers and copolymers of acrylic acid and methacrylic acid and salts thereof. These also include crosslinked polymers of acrylic acid, as are available under the INCI name Carbomer. Such crosslinked homopolymers of acrylic acid are, for example, obtainable commercially under the name Carbopol® from BF GOODRICH. Preference is also given to hydrophobically modified crosslinked polyacrylate polymers, such as Carbopol® Ultrez 21 from Noveon.

Compositions based on homopolymers and copolymers of acrylic acid and methacrylic acid are suitable in an advantageous manner for formulation as gels, for example for setting gels, and for formulation of foams.

Further examples of suitable anionic polymers are copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of (meth)acrylic acid and polyether acrylates, the polyether chain being terminated with a C₈-C₃₀-alkyl radical. These include, for example, acrylate/beheneth-25 methacrylate copolymers which are obtainable under the name Aculyn® from Rohm and Haas. Particularly suitable polymers are also copolymers of t-butyl acrylate, ethyl acrylate, methacrylic acid (e.g. Luvimer® 100P), copolymers of ethyl acrylate and methacrylic acid (e.g. Luvimer® MAE), copolymers of N-tert-butylacrylamide, ethyl acrylate, acrylic acid (Ultrahold® 8, strong), copolymers of vinyl acetate, crotonic acid and optionally further vinyl esters (e.g. Luviset® brands), maleic anhydride copolymers, if appropriate reacted with alcohol, anionic polysiloxanes, e.g. carboxy functional ones, t-butyl acrylate, methacrylic acid (e.g. Luviskol® VBM), copolymers of acrylic acid and methacrylic acid with hydrophobic monomers, such as, for example, C₄-C₃₀-alkyl esters of meth(acrylic acid), C₄-C₃₀-alkyl vinyl esters, C₄-C₃₀-alkyl vinyl ethers and hyaluronic acid. Examples of anionic polymers are also vinyl acetate/crotonic acid copolymers, as for sale, for example, under the names Resyn® (National Starch) and Gafset® (GAF) and vinylpyrrolidone/vinyl acrylate copolymers obtainable, for example, under the trade name Luviflex® (BASF). Further suitable polymers are the vinylpyrrolidone/acrylate terpolymer available under the name Luviflex® VBM-35 (BASF) and sodium sulfonate-containing polyamides or sodium sulfonate-containing polyesters.

In addition, the group of suitable anionic polymers comprises, by way of example, Balance® CR (National Starch; acrylate copolymer), Balance® 0/55 (National Starch; acrylate copolymer), Balance® 47 (National Starch; octylacrylamide/acrylate/-butylaminoethyl methacrylate copolymer), Aquaflex® FX 64 (ISP; isobutylene/-ethylmaleimide/hydroxyethylmaleimide copolymer), Aquaflexe SF-40 (ISP/National Starch; VP/vinyl caprolactam/DMAPA acrylate copolymer), Allianz® LT-120 (ISP/Rohm & Haas; acrylate/C₁₋₂ succinate/hydroxyacrylate copolymer), Aquarez® HS (Eastman; polyester-1), Diaformer® Z-400 (Clariant; methacryloylethylbetaine/methacrylate copolymer), Diaformer® Z-711 (Clariant; methacryloylethyl N-oxide/methacrylate copolymer), Diaformer® Z-712 (Clariant; methacryloylethyl N-oxide/methacrylate copolymer), Omnirez® 2000 (ISP; monoethylester of poly(methyl vinyl ether/maleic acid in ethanol), Amphomer® HC (National Starch; acrylate/octylacrylamide copolymer), Amphomer® 28-4910 (National Starch; octylacrylamide/acrylate/butyl-aminoethyl methacrylate copolymer), Advantage® HC 37 (ISP; terpolymer of vinylcaprolactam/vinylpyrrolidone/dimethylaminoethyl methacrylate), Advantage® LC55 and LC80 or LC A and LC E, Advantage® Plus (ISP; VA/butyl maleate/isobornyl acrylate copolymer), Acudyne® 258 (Rohm & Haas; acrylate/hydroxyester acrylate copolymer), Luviset® P.U.R. (BASF, polyurethane-1), Luviflex® Silk (BASF), Eastman® AQ 48 (Eastman), Styleze®CC-10 (ISP; VP/DMAPA acrylates copolymer), Styleze® 2000 (ISP; VP/acrylates/lauryl methacrylate copolymer), DynamX (National Starch; polyurethane-14 AMP-acrylates copolymer), Resyn XP (National Starch; acrylates/octylacrylamide copolymer), Fixomer A-30 (Ondeo Nalco; polymethacrylic acid (and) acrylamidomethylpropanesulfonic acid), Fixate G-100 (Noveon; AMP-acrylates/allyl methacrylate copolymer).

Suitable copolymers S) are also the terpolymers of vinylpyrrolidone, C₁-C₁₀-alkyl, cycloalkyl and aryl (meth)acrylates and acrylic acid described in U.S. Pat. No. 3,405,084. Suitable copolymers S) are also the terpolymers of vinylpyrrolidone, tert-butyl (meth)acrylate and (meth)acrylic acid described in EP-A-0 257 444 and EP-A-0 480 280. Suitable copolymers A2) are also the copolymers described in DE-A-42 23 066 which comprise at least one (meth)acrylic ester, (meth)acrylic acid, and N-vinylpyrrolidone and/or N-vinylcaprolactam in copolymerized form. The disclosure of these documents is hereby incorporated by reference.

The abovementioned polymers S) are prepared by known methods, for example solution, precipitation, suspension or emulsion polymerization.

Suitable carboxylic acid group-containing polymers S) are also carboxylic acid group-containing polyurethanes.

EP-A-636 361 discloses suitable block copolymers with polysiloxane blocks and polyurethane/polyurea blocks which have carboxylic acid and/or sulfonic acid groups. Suitable silicone-containing polyurethanes are also described in WO 97/25021 and EP-A-751 162.

Suitable polyurethanes are also described in DE-A-42 25 045, which is hereby incorporated by reference in its entirety. These polyurethanes are in principle composed of
i) at least one compound which comprises two or more active hydrogen atoms per molecule,
ii) at least one carboxylic acid group-containing diol or a salt thereof and
iii) at least one polyisocyanate.

Component i) is, for example, a diol, diamine, amino alcohol, or mixture thereof. The molecular weight of these compounds is preferably in a range from about 56 to 280. If desired, up to 3 mol % of said compounds can be replaced by triols or triamines.

Diols i) which can be used are, for example, ethylene glycol, propylene glycol, butylene glycol, neopentyl glycol, cyclohexanedimethylol, di-, tri-, tetra-, penta- or hexaethylene glycol and mixtures thereof. Preference is given to using neopentyl glycol and/or cyclohexanedimethylol. Suitable aminoalcohols i) are, for example, 2-aminoethanol, 2-(N-methylamino)ethanol, 3-aminopropanol, 4-aminobutanol, 1-ethylaminobutan-2-ol, 2-amino-2-methyl-1-propanol, 4-methyl-4-aminopentan-2-ol etc. Suitable diamines i) are, for example, ethylenediamine, propylenediamine, 1,4-diaminobutane, 1,5-diaminopentane and 1,6-diaminohexane, and α,ω-diaminopolyethers, which can be prepared by amination of polyalkylene oxides with ammonia.

Component i) may also be a polymer with a number-average molecular weight in the range from about 300 to 5000, preferably about 400 to 4000, in particular 500 to 3000. Polymers i) which can be used are, for example, polyesterdiols, polyetherols and mixtures thereof. Polyetherols are preferably polyalkylene glycols, e.g. polyethylene glycols, polypropylene glycols, polytetrahydrofurans etc., block copolymers of ethylene oxide and propylene oxide or block copolymers of ethylene oxide, propylene oxide and butylene oxide which comprise the copolymerized alkylene oxide units in random distribution or in the form of blocks. Suitable polytetrahydrofurans i) can be prepared by cationic polymerization of tetrahydrofuran in the presence of acidic catalysts, such as, for example, sulfuric acid or fluorosulfuric acid. Such production methods are known to the person skilled in the art. Polyesterdiols i) which can be used preferably have a number-average molecular weight in the range from about 400 to 5000, preferably 500 to 3000, in particular 600 to 2000. Suitable polyesterdiols i) are all those which are customarily used for producing polyurethanes, in particular those based on aromatic dicarboxylic acids, such as terephthalic acid, isophthalic acid, phthalic acid, Na or K sulfoisophthalic acid etc., aliphatic dicarboxylic acids, such as adipic acid or succinic acid etc., and cycloaliphatic dicarboxylic acids, such as 1,2-, 1,3- or 1,4-cyclohexanedicarboxylic acid. Suitable diols are, in particular, aliphatic diols, such as ethylene glycol, propylene glycol, 1,6-hexanediol, neopentyl glycol, diethylene glycol, polyethylene glycols, polypropylene glycols, 1,4-dimethylolcyclohexane, etc.

Suitable compounds ii) which have two active hydrogen atoms and at least one carboxylic acid group per molecule are, for example, dimethylolpropanoic acid and mixtures which comprise dimethylolpropanoic acid.

Component iii) is a customary aliphatic, cycloaliphatic and/or aromatic polyisocyanate, such as tetramethylene diisocyanate, hexamethylene diisocyanate, methylenediphenyl diisocyanate, 2,4- and 2,6-tolylene diisocyanate and isomer mixtures thereof, o- and m-xylylene diisocyanate, 1,5-naphthylene diisocyanate, 1,4-cyclohexylene diisocyanate, dicyclohexylmethane diisocyanate and mixtures thereof, in particular isophorone diisocyanate and/or dicyclohexylmethane diisocyanate. If desired, up to 3 mol % of said compounds can be replaced by triisocyanates.

The polyurethane polymers S) are produced by customary methods known to the person skilled in the art, as described above for the urethane compounds A) according to the invention.

The invention further provides a cosmetic or pharmaceutical composition comprising
A) at least one urethane compound as defined above and
B) at least one cosmetically or pharmaceutically acceptable carrier.

### Cosmetically Acceptable Carrier B)

The compositions according to the invention have a cosmetically or pharmaceutically acceptable carrier B), which is preferably chosen from
i) water,
ii) water-miscible organic solvents, preferably C₂-C₄-alkanols, in particular ethanol,
iii) oils, fats, waxes,
iv) esters of C₆-C₃₀-monocarboxylic acids with mono-, di- or trihydric alcohols which are different from iii),
v) saturated acyclic and cyclic hydrocarbons,
vi) fatty acids,
vii) fatty alcohols,
viii) propellant gases,
   and mixtures thereof.

Suitable hydrophilic carriers B) are chosen from water, 1-, 2- or polyhydric alcohols having preferably 1 to 8 carbon atoms, such as ethanol, n-propanol, isopropanol, propylene glycol, glycerol, sorbitol, etc.

Suitable propellant gases B) are the propellants customarily used, for example, for hairsprays or aerosol foams. Preference is given to mixtures of propane/butane, pentane, dimethyl ether, 1,1-difluoroethane (HFC-152a), carbon dioxide, nitrogen or compressed air.

Suitable cosmetically and pharmaceutically compatible oil and/or fat components B) are described in Karl-Heinz Schrader, Grundlagen und Rezepturen der Kosmetika [Fundamentals and formulations of cosmetics], 2^{nd} edition, Verlag Hüthig, Heidelberg, pp. 319-355, which is incorporated herein by reference.

The compositions according to the invention have, for example, an oil and/or fat component B) which is chosen from: hydrocarbons of low polarity; linear saturated hydrocarbons; cyclic hydrocarbons; branched hydrocarbons; animal and vegetable oils; waxes; wax esters; Vaseline; esters of fatty acids; salicylates; benzoate esters, etc. and mixtures thereof.

Suitable carriers B) are also silicone oils, e.g. linear polydimethylsiloxanes, poly(methylphenylsiloxanes), cyclic siloxanes and mixtures thereof.

Suitable oil and/or fat components B) are also paraffin and paraffin oils; vaseline; natural fats and oils; fatty alcohols; fatty acids; waxes, and mixtures of the abovementioned oil and/or fat components.

The cosmetic compositions according to the invention may be skin cosmetic, hair cosmetic, dermatological, hygiene or pharmaceutical compositions. On account of their properties, the polyurethane compounds A) described above are particularly suitable as additives for hair and skin cosmetics.

Preference is given to compositions which additionally comprise at least one acid group-containing polymer S).

Preferably, the compositions according to the invention are in the form of a gel, foam, spray, ointment, cream, emulsion, suspension, lotion, milk or paste. If desired, liposomes or microspheres can also be used.

The cosmetically or pharmaceutically active compositions according to the invention can additionally comprise cosmetically and/or dermatologically active ingredients and auxiliaries.

Preferably, the cosmetic compositions according to the invention comprise at least one urethane compound A) as defined above, at least one carrier B) as defined above, if appropriate at least one acid group-containing polymer C) and at least one constituent different therefrom which is preferably chosen from cosmetically active ingredients, emulsifiers, surfactants, preservatives, perfume oils, thickeners, hair polymers, hair and skin conditioners, graft polymers, water-soluble or dispersible silicone-containing polymers, photoprotective agents, bleaches, gel formers, care agents, colorants, tints, tanning agents, dyes, pigments, consistency regulators, humectants, regreasing agents, collagen, protein hydrolyzates, lipids, antioxidants, antifoams, antistats, emollients and softeners.

Customary thickeners in such formulations are crosslinked polyacrylic acids and derivatives thereof, polysaccharides and derivatives thereof, such as xanthan gum, agar agar, alginates or tyloses, cellulose derivatives, e.g. carboxymethylcellulose or hydroxycarboxymethylcellulose, fatty alcohols, monoglycerides and fatty acids, polyvinyl alcohol and polyvinylpyrrolidone. Preference is given to using nonionic thickeners.

Suitable cosmetically and/or dermatologically active ingredients are, for example, coloring active ingredients, skin and hair pigmentation agents, tints, tanning agents, bleaches, keratin-hardening substances, antimicrobial active ingredients, photofilter active ingredients, repellent active ingredients, substances with hyperemic activity, substances with keratolytic and keratoplastic activity, antidandruff active ingredients, antiphlogistics, substances which have a keratinizing effect, substances which act as antioxidants or as free-radical scavengers, skin moisturizers or humectants, regreasing active ingredients, antierythimatous or antiallergic active ingredients and mixtures thereof.

Artificially skin-tanning active ingredients which are suitable for tanning the skin without natural or artificial irradiation with UV rays are, for example, dihydroxyacetone, alloxan and walnut shell extract. Suitable keratin-hardening substances are usually active ingredients as are also used in antiperspirants, such as, for example, potassium aluminum sulfate, aluminum hydroxychloride, aluminum lactate, etc. Antimicrobial active ingredients are used in order to destroy microorganisms or to inhibit their growth and thus serve both as preservatives and also as a deodorizing substance which reduces the formation or the intensity of body odor. These include, for example, customary preservatives known to the person skilled in the art, such as p-hydroxybenzoates, imidazolidinylurea, formaldehyde, sorbic acid, benzoic acid, salicylic acid, etc. Such deodorizing substances are, for example, zinc ricinoleate, triclosan, undecylenic acid alkylolamides, triethyl citrate, chlorhexidine etc. Suitable photofilter active ingredients are substances which absorb UV rays in the UV-B and/or UV-A region. Suitable UV filters are, for example, 2,4,6-triaryl-1,3,5-triazines in which the aryl groups may each carry at least one substituent which is preferably chosen from hydroxyl, alkoxy, specifically methoxy, alkoxycarbonyl, specifically methoxycarbonyl and ethoxycarbonyl and mixtures thereof. Also suitable are p-aminobenzoates, cinnamates, benzophenones, camphor derivatives, and pigments which stop UV rays, such as titanium dioxide, talc and zinc oxide. Suitable repellent active ingredients are compounds which are able to drive away or repel certain animals, in particular insects, from humans. These include, for example, 2-ethyl-1,3-hexanediol, N,N-diethyl-m-toluamide etc. Suitable substances with hyperemic activity, which stimulate blood flow through the skin, are, for example, ethereal oils, such as dwarf pine, lavender, rosemary, juniper berry, horse chestnut extract, birch leaf extract, hayflower extract, ethyl acetate, camphor, menthol, peppermint oil, rosemary extract, eucalyptus oil, etc. Suitable substances with a keratolytic and keratoplastic effect are, for example, salicylic acid, calcium thioglycolate, thioglycolic acid and its salts, sulfur, etc. Suitable antidandruff active ingredients are, for example, sulfur, sulfur polyethylene glycol sorbitan monooleate, sulfur ricinol polyethoxylate, zinc pyrithione, aluminum pyrithione, etc. Suitable antiphlogistics which counter skin irritations are, for example, allantoin, bisabolol, dragosantol, chamomile extract, panthenol, etc.

The cosmetic compositions according to the invention can comprise, as cosmetic and/or pharmaceutical active ingredient (and also if appropriate as auxiliary), at least one cosmetically or pharmaceutically acceptable polymer which differs from the polymers A) and C). Very generally, these include cationic, amphoteric and neutral polymers.

Suitable polymers are, for example, cationic polymers with the INCI name Polyquaternium, e.g. copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinyl-imidazolium salts (Luviquat® Hold); cationic cellulose derivatives (Polyquaternium-4 and -10), acrylamidocopolymers (Polyquaternium-7) and chitosan. Suitable cationic (quaternized) polymers are also Merquat® (polymer based on dimethyldiallyl-ammonium chloride), Gafquat® (quaternary polymers which are formed by reacting polyvinylpyrrolidone with quaternary ammonium compounds), polymer JR (hydroxyethylcellulose with cationic groups) and plant-based cationic polymers, e.g. guar polymers, such as the Jaguar® grades from Rhodia.

Further suitable polymers are also neutral polymers, such as polyvinylpyrrolidones, copolymers of N-vinylpyrrolidone and vinyl acetate and/or vinyl propionate, polysiloxanes, polyvinylcaprolactam and other copolymers with N-vinylpyrrolidone, polyethyleneimines and salts thereof, polyvinylamines and salts thereof, cellulose derivatives, polyaspartic acid salts and derivatives. These include, for example, Luviflex® Swing (partially saponified copolymer of polyvinyl acetate and polyethylene glycol, BASF).

Suitable polymers are also nonionic, water-soluble or water-dispersible polymers or oligomers, such as polyvinylcaprolactam, e.g. Luviskol® Plus (BASF), or polyvinylpyrrolidone and copolymers thereof, in particular with vinyl esters, such as vinyl acetate, e.g. Luviskol® VA 37 (BASF); polyamides, e.g. based on itaconic acid and aliphatic diamines, as described, for example, in DE-A-43 33 238.

Suitable polymers are also amphoteric or zwitterionic polymers, such as the octylacrylamide/methyl methacrylate/tert-butylaminoethyl methacrylate/2-hydroxypropyl methacrylate copolymers obtainable under the names Amphomer® (National Starch), and zwitterionic polymers, as are disclosed, for example, in the German patent applications DE 39 29 973, DE 21 50 557, DE 28 17 369 and DE 37 08 451. Acrylamidopropyltrimethylammonium chloride/acrylic acid or methacrylic acid copolymers and the alkali metal and ammonium salts thereof are preferred zwitterionic polymers. Further suitable zwitterionic polymers are methacroylethylbetaine/-methacrylate copolymers, which are available commercially under the name Amersette® (AMERCHOL), and copolymers of hydroxyethyl methacrylate, methyl methacrylate, N,N-dimethylaminoethyl methacrylate and acrylic acid (Jordapon®).

Suitable polymers are also nonionic, siloxane-containing, water-soluble or -dispersible polymers, e.g. polyether siloxanes, such as Tegopren® (Goldschmidt) or Belsil® (Wacker).

The formulation base of pharmaceutical compositions according to the invention preferably comprises pharmaceutically acceptable auxiliaries. Pharmaceutically acceptable auxiliaries are the auxiliaries which are known for use in the fields of pharmacy, food technology and related fields, in particular the auxiliaries listed in the relevant pharmacopeias (e.g. DAB Ph. Eur. BP NF), and other auxiliaries whose properties do not preclude a physiological application.

Suitable auxiliaries may be: glidants, wetting agents, emulsifying and suspending agents, preservatives, antioxidants, antiirritative substances, chelating agents, emulsion stabilizers, film formers, gel formers, odor-masking agents, resins, hydrocolloids, solvents, solubility promoters, neutralizing agents, permeation accelerators, pigments, quaternary ammonium compounds, regressing and supergreasing agents, ointment bases, cream bases or oil bases, silicone derivatives, stabilizers, sterilizing agents, propellants, drying agents, opacifiers, thickeners, waxes, softeners, white oils. Formulation in this regard is based on expert knowledge, as given, for example, in Fiedler, H. P. Lexikon der Hilfsstoffe fOr Pharmazie, Kosmetik und angrenzende Gebiete [Lexicon of auxiliaries for pharmacy, cosmetics and related fields], 5^{th} edition, Aulendorf: ECV-Editio-Cantor-Verlag, 2002.

To prepare the dermatological compositions according to the invention the active ingredients can be mixed or diluted with a suitable auxiliary (excipient). Excipients may be solid, semisolid or liquid materials which can serve as a vehicle, carrier or medium for the active ingredient. The admixing of further auxiliaries takes place if desired in the manner known to the person skilled in the art. In addition, the polymers P) and dispersions Pd) are suitable as auxiliaries in pharmacy, preferably as or in (a) coating(s) or binder(s) for solid medicaments. They can also be used in creams and as tablet coatings and tablet binders.

According to a preferred embodiment, the compositions according to the invention are a skin-cleansing composition.

Preferred skin-cleansing compositions are soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, shower and bath preparations, such as washing lotions, shower baths and shower gels, foam baths, oils baths and scrub preparations, shaving foams, shaving lotions and shaving creams.

According to a further preferred embodiment, the compositions according to the invention are cosmetic compositions for the care and protection of the skin, nail care compositions or preparations for decorative cosmetics.

Suitable skin cosmetic compositions are, for example, face tonics, face masks, deodorants and other cosmetic lotions. Compositions for use in decorative cosmetics comprise, for example, concealing sticks, stage make-up, mascara and eyeshadows, lipsticks, kohl pencils, eyeliners, blushers, powders and eyebrow pencils.

Furthermore, the urethane compounds A) can be used in nose strips for pore cleansing, in antiacne compositions, repellents, shaving compositions, depilatories, intimate care compositions, footcare compositions and in babycare.

The skincare compositions according to the invention are, in particular, W/O or O/W skin creams, day and night creams, eye creams, face creams, antiwrinkle creams, moisturizing creams, bleaching creams, vitamin creams, skin lotions, care lotions and moisturizing lotions.

Skin cosmetic and dermatological compositions based on the above-described urethane compounds A) exhibit advantageous effects. The polymers can, inter alia, contribute to the moisturizing and conditioning of the skin and to an improvement in the skin sensation. The polymers can also act as thickeners in the formulations. By adding the polymers according to the invention, it is possible to achieve a considerable improvement in skin compatibility in certain formulations.

Skin cosmetic and dermatological compositions preferably comprise at least one urethane compound A) in an amount of from about 0.001 to 30% by weight, preferably 0.01 to 20% by weight, very particularly preferably 0.1 to 12% by weight, based on the total weight of the composition.

Photoprotective agents based on the urethane compounds A), in particular, have the property of increasing the residence time of the UV-absorbing ingredients compared with customary auxiliaries such as polyvinylpyrrolidone.

Depending on the field of use, the compositions according to the invention can be applied in a form suitable for skincare, such as, for example, as cream, foam, gel, stick, mousse, milk, spray (pump spray or spray containing propellant) or lotion.

Besides the urethane compounds A) and suitable carriers, the skin cosmetic preparations can also comprise further active ingredients and auxiliaries customary in skin cosmetics, as described above. These include, preferably, emulsifiers, preservatives, perfume oils, cosmetic active ingredients, such as phytantriol, vitamin A, E and C, retinol, bisabolol, panthenol, photoprotective agents, bleaches, colorants, tinting agents, tanning agents, collagen, protein hydrolyzates, stabilizers, pH regulators, dyes, salts, thickeners, gel formers, consistency regulators, silicones, moisturizers, regreasing agents and further customary additives.

Preferred oil and fat components of the skin cosmetic and dermatological compositions are the abovementioned mineral and synthetic oils, such as, for example, paraffins, silicone oils and aliphatic hydrocarbons with more than 8 carbon atoms, animal and vegetable oils, such as, for example, sunflower oil, coconut oil, avocado oil, olive oil, lanolin, or waxes, fatty acids, fatty acid esters, such as, for example, triglycerides of C₆-C₃₀-fatty acids, wax esters, such as, for example, jojoba oil, fatty alcohols, vaseline, hydrogenated lanolin and acetylated lanolin, and mixtures thereof.

The polymers according to the invention can also be mixed with conventional polymers where specific properties are to be set.

To set certain properties, such as, for example, improving the feel to the touch, the spreading behavior, the water resistance and/or the binding of active ingredients and auxiliaries, such as pigments, the skin cosmetic and dermatological preparations can comprise additional conditioning substances based on silicone compounds besides the urethane compounds A). Suitable silicone compounds are, for example, polyalkylsiloxanes, polyarylsiloxanes, polyarylalkylsiloxanes, polyether siloxanes or silicone resins. Use of the urethane compounds according to the invention often renders the use of such additional silicone compounds superfluous.

The cosmetic or dermatological preparations are prepared by customary methods known to the person skilled in the art.

The cosmetic and dermatological compositions are preferably in the form of emulsions, in particular water-in-oil (WIO) emulsions or oil-in-water (O/W) emulsions. It is, however, also possible to choose other types of formulation, for example hydrodispersions, gels, oils, oleogels, multiple emulsions, for example in the form of W/O/W or O/W/O emulsions, anhydrous ointments or ointment bases, etc.

The emulsions are prepared by known methods. Besides at least one urethane compound A), the emulsions usually comprise customary constituents, such as fatty alcohols, fatty acid esters and, in particular, fatty acid triglycerides, fatty acids, lanolin and derivatives thereof, natural or synthetic oils or waxes and emulsifiers in the presence of water. The choice of emulsion type-specific additives and the preparation of suitable emulsifiers is described, for example, in Schrader, Grundlagen und Rezepturen der Kosmetika [Fundamentals and formulations of cosmetics], HOthig Buch Verlag, Heidelberg, 2^{nd} edition, 1989, third part, which is hereby expressly incorporated by reference.

A suitable emulsion, e.g. for a skin cream etc., generally comprises an aqueous phase which is emulsified by means of a suitable emulsifier system in an oil or fatty phase. To provide the aqueous phase, a urethane compound A) can be used.

Preferred fatty components which the fatty phase of the emulsions may comprise are: hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, caratea oil, hoplostethus oil; mineral oils whose distillation start-point under atmospheric pressure is about 250° C. and whose distillation end-point is 410° C., such as, for example, Vaseline oil; esters of saturated or unsaturated fatty acids, such as alkyl myristates, e.g. i-propyl, butyl or cetyl myristate, hexadecyl stearate, ethyl or isopropyl palmitate, octanoic or decanoic acid triglycerides and cetyl ricinoleate.

The fatty phase may also comprise silicone oils soluble in other oils, such as dimethylpolysiloxane, methylphenylpolysiloxane and the silicone glycol copolymer, fatty acids and fatty alcohols.

Besides the urethane compounds A), it is also possible to use waxes, such as, for example, carnauba wax, candelilla wax, beeswax, microcrystalline wax, ozokerite wax and the oleates, myristates, linoleates and stearates of Ca, Mg and Al.

In addition, an emulsion according to the invention may be in the form of an O/W emulsion. Such an emulsion usually comprises an oil phase, emulsifiers which stabilize the oil phase in the water phase, and an aqueous phase which is usually present in thickened form. Suitable emulsifiers are preferably O/W emulsifiers, such as polyglycerol esters, sorbitan esters or partially esterified glycerides.

According to a further preferred embodiment, the compositions according to the invention are a shower gel, a shampoo formulation or a bath preparation.

Such formulations comprise at least one urethane compound A) and customary anionic surfactants as base surfactants and amphoteric and/or nonionic surfactants as cosurfactants. Other suitable active ingredients and/or auxiliaries are generally chosen from lipids, perfume oils, dyes, organic acids, preservatives and antioxidants, and thickeners/gel formers, skin conditioning agents and humectants.

These formulations preferably comprise 2 to 50% by weight, preferably 5 to 40% by weight, particularly preferably 8 to 30% by weight, of surfactants, based on the total weight of the formulation.

All anionic, neutral, amphoteric or cationic surfactants customarily used in body-cleansing compositions can be used in the washing, shower and bath preparations.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

These include, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropyl-betaines, alkylsulfobetaines, alkyl glycinates, alkyl carboxyglycinates, alkyl amphoacetates or amphopropionates, alkyl amphodiacetates or amphodipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 moles per mol of alcohol. Also suitable are alkylamine oxides, mono- or dialkylalkanolamides, fatty acid esters of polyethylene glycols, ethoxylated fatty acid amides, alkyl polyglycosides or sorbitan ether esters.

Furthermore, the washing, shower and bath preparations can also comprise customary cationic surfactants such as, for example, quaternary ammonium compounds, for example cetyltrimethylammonium chloride.

In addition, the shower gel/shampoo formulations can comprise thickeners, such as, for example, sodium chloride, PEG-55, propylene glycol oleate, PEG-120 methylglucose dioleate and others, and also preservatives, further active ingredients and auxiliaries and water.

According to a further preferred embodiment, the compositions according to the invention are hair-treatment compositions.

Hair-treatment compositions according to the invention preferably comprise at least one urethane compound A) in an amount in the range from about 0.1 to 30% by weight, preferably 0.5 to 20% by weight, based on the total weight of the composition.

The hair-treatment compositions according to the invention are preferably in the form of a setting foam, hair mousse, hair gel, shampoo, hairspray, hair foam, end fluid, neutralizer for permanent waves, hair colorant and bleach or “hot-oil treatment”. Depending on the field of use, the hair cosmetic preparations can be applied in the form of an (aerosol) spray, (aerosol) foam, gel, gel spray, cream, lotion or wax. Hairsprays comprise here both aerosol sprays and also pump sprays without propellant gas. Hair foams comprise both aerosol foams and also pump foams without propellant gas. Hairsprays and hair foams preferably comprise predominantly or exclusively water-soluble or water-dispersible components. If the compounds used in the hairsprays and hair foams according to the invention are water-dispersible, they can be used in the form of aqueous microdispersions with particle diameters of customarily 1 to 350 nm, preferably 1 to 250 nm. The solids contents of these preparations are usually in a range from about 0.5 to 20% by weight. These microdispersions generally require no emulsifiers or surfactants for their stabilization.

In a preferred embodiment, the hair cosmetic formulations according to the invention comprise
0.05 to 20% by weight of at least one urethane compound A),
0 to 50% by weight of at least one acid group-containing polymer C),
20 to 99.95% by weight of water and/or alcohol,
0 to 50% by weight of at least one propellant gas,
0 to 5% by weight of at least one emulsifier,
0 to 3% by weight of at least one thickener, and
up to 25% by weight of further constituents.

Alcohol is understood as meaning all alcohols customary in cosmetics, e.g. ethanol, isopropanol, n-propanol.

Further constituents are understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances, such as panthenol, collagen, vitamins, protein hydrolyzates, alpha- and beta-hydroxycarboxylic acids, protein hydrolyzates, stabilizers, pH regulators, dyes, viscosity regulators, gel formers, dyes, salts, moisturizers, regreasing agents, complexing agents and further customary additives.

Also included here are all styling and conditioner polymers known in cosmetics which can be used in combination with the polymers according to the invention if quite specific properties are to be set.

Suitable conventional hair cosmetic polymers are, for example, the abovementioned cationic, anionic, neutral, nonionic and amphoteric polymers, which are hereby incorporated by reference.

To set certain properties, the preparations can additionally also comprise conditioning substances based on silicone compounds. Suitable silicone compounds are, for example, polyalkylsiloxanes, polyarylsiloxanes, polyarylalkylsiloxanes, polyether siloxanes, silicone resins or dimethicone copolyols (CTFA) and aminofunctional silicone compounds, such as amodimethicone (CTFA).

The polymers according to the invention are particularly suitable as conditioner agents and/or setting agents in hairstyling preparations, in particular hairsprays (aerosol sprays and pump sprays without propellant gas) and hair foams (aerosol foams and pump foams without propellant gas).

In a preferred embodiment, spray preparations comprise
0.1 to 10% by weight of at least one urethane compound A),
0 to 30% by weight of at least one acid group-containing polymer C),
20 to 99.9% by weight of water and/or alcohol,
0 to 70% by weight of at least one propellant,
0 to 20% by weight of further constituents.

Propellants are the propellants used customarily for hairsprays and aerosol foams. Preference is given to mixtures of propane/butane, pentane, dimethyl ether, 1,1-difluoroethane (HFC-152 a), carbon dioxide, nitrogen or compressed air.

A formulation preferred according to the invention for aerosol hair foams comprises
0.1 to 10% by weight of at least one urethane compound A),
0 to 30% by weight of at least one acid group-containing polymer C),
55 to 99.8% by weight of water and/or alcohol,
5 to 20% by weight of a propellant,
0.1 to 5% by weight of an emulsifier,
0 to 10% by weight of further constituents.

The emulsifiers used may be all emulsifiers customarily used in hair foams. Suitable emulsifiers may be nonionic, cationic or anionic or amphoteric.

Examples of nonionic emulsifiers (INCI nomenclature) are laureths, e.g. laureth-4; ceteths, e.g. ceteth-1, polyethylene glycol cetyl ethers; ceteareths, e.g. ceteareth-25, polyglycol fatty acid glycerides, hydroxylated lecithin, lactyl esters of fatty acids, alkyl polyglycosides.

Examples of cationic emulsifiers are cetyidimethyl-2-hydroxyethylammonium dihydrogenphosphate, cetyltrimonium chloride, cetyltrimonium bromide, cocotrimonium methyl sulfate, quaternium-1 to x (INCI).

Anionic emulsifiers may be chosen, for example, from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the following composition:
0.1 to 10% by weight of at least one urethane compound A),
0 to 30% by weight of at least one acid group-containing polymer C),
80 to 99.85% by weight of water and/or alcohol,
0 to 3% by weight, preferably 0.05 to 2% by weight, of a gel former,
0 to 20% by weight of further constituents.

In the preparation of gels based on the urethane compounds A) according to the invention, gel formers can be used, for example, in order to establish specific rheological or other application properties of the gels. Gel formers which may be used are all gel formers customary in cosmetics. These include slightly crosslinked polyacrylic acid, for example carbomer (INCI), cellulose derivatives, e.g. hydroxypropylcellulose, hydroxyethylcellulose, cationically modified celluloses, polysaccharides, e.g. xanthan gum, caprylic/capric triglyceride, sodium acrylate copolymers, polyquaternium-32 (and) paraffinum liquidum (INCI), sodium acrylate copolymers (and) paraffinum liquidum (and) PPG-1 trideceth-6, acrylamidopropyl-trimonium chloride/acrylamide copolymers, steareth-10 allyl ether acrylate copolymers, polyquaternium-37 (and) paraffinum liquidum (and) PPG-1 trideceth-6, polyquaternium 37 (and) propylene glycol dicaprate dicaprylate (and) PPG-1 trideceth-6, polyquaternium-7, polyquaternium44.

Crosslinked homopolymers of acrylic acid suitable as gel formers are commercially available, for example, under the name Carbopol® from BF GOODRICH. Preference is also given to hydrophobically modified crosslinked polyacrylate polymers, such as Carbopol® Ultrez 21 from Noveon. Further examples of anionic polymers suitable as gel formers are copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of (meth)acrylic acid and polyether acrylates where the polyether chain is terminated with a C₈-C₃₀-alkyl radical. These include, for example, acrylate/beheneth-25 methacrylate copolymers, which are available under the name Aculyn® from Rohm & Haas.
The polyurethanes A) according to the invention can be used as conditioners in cosmetic preparations.

The polyurethanes A) according to the invention can preferably be used in shampoo formulations as setting agents and/or conditioners. Preferred shampoo formulations comprise
0.05 to 10% by weight of at least one urethane compound A),
25 to 94.95% by weight of water,
5 to 50% by weight of surfactants,
0 to 5% by weight of a further conditioner,
0 to 10% by weight of further cosmetic constituents.

In the shampoo formulations it is possible to use all anionic, neutral, amphoteric or cationic surfactants customarily used in shampoos.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

For example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate are suitable.

Suitable amphoteric surfactants are, for example, alkylbetaines, alkylamidopropyl-betaines, alkylsulfobetaines, alkyl glycinates, alkyl carboxyglycinates, alkyl amphoacetates or amphopropionates, alkyl amphodiacetates or dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 moles per mole of alcohol. In addition, alkylamine oxides, mono- or dialkylalkanolamides, fatty acid esters of polyethylene glycols, alkyl polyglycosides or sorbitan ether esters are suitable.

Furthermore, the shampoo formulations can comprise customary cationic surfactants, such as, for example, quaternary ammonium compounds, for example cetyltrimethyl-ammonium chloride.

In the shampoo formulations, in order to achieve certain effects, customary conditioners can be used in combination with the crosslinked polyurethanes. These include, for example, the abovementioned cationic polymers with the INCI name Polyquaternium, in particular copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/-N-vinylimidazolium salts (Luviquat® Hold); cationic cellulose derivatives (Polyquaternium-4 and -10), acrylamide copolymers (Polyquaternium-7). It is also possible to use protein hydrolyzates, and conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and aminofunctional silicone compounds, such as amodimethicone (CTFA). It is also possible to use cationic guar derivatives, such as Guar Hydroxypropyltrimonium Chloride (INCI).

The invention further provides the use of a urethane compound as defined above in skin-cleansing compositions, compositions for the protection and the care of the skin, nail care compositions, preparations for decorative cosmetics and in hair-treatment compositions.

The invention further provides the use of a urethane compound A) as defined above as auxiliary in pharmacy, preferably as or in (a) coating(s) for solid drug forms, for modifying rheological properties, as surface-active compound, as or in (an) adhesive(s), and as or in (a) coating(s) for the textile, paper, printing and leather industry.

The invention is explained in more detail by reference to the following nonlimiting examples.

### EXAMPLES

### I. Production of Urethane Compounds

### Example 1

| | | | |
|---|---|---|---|
| Initial charge | 180 | g (0.02 mol) | ethoxylated/propoxylated |
| | | | polydimethyl-siloxane, |
| | | | Mₙ = 9000 g/mol |
| | | | (Pluriol ® ST 4005, BASF |
| | | | Aktiengesell-schaft) |
| Feed 1 | 27.7 | g (0.125 mol) | Isophorone diisocyanate |
| Feed 2 | 50.0 | g | Ethanol |
| Feed 3 | 9.2 | g (0.135 mol) | Imidazole |
| Feed 4 | 160 | g | Ethanol |

In a 4-necked flask which was equipped with stirrer, dropping funnel, thermometer, reflux condenser and a device for working under nitrogen, the initial charge was heated to an external temperature of 85° C. with stirring. After an internal temperature of 50° C. had been reached, feed 1 was added over the course of 10 min. The reaction mixture obtained was stirred for a further 3 h at an internal temperature of 80° C. The mixture was then left to cool to 30° C., and feed 2 was added and homogenized. This gave a polyurethane prepolymer with an NCO content of about 3%. At an internal temperature of 20° C., feed 3 was metered in over the course of 10 min and the resulting reaction mixture was then stirred for a further 30 min at a temperature of 40° C. The polymer mass obtained in this way was diluted by adding feed 4 at a temperature of 40° C. This gave a pale yellow clear viscous solution with a solids content of about 50%. The product had a K value (measured on a 1% strength solution N-methylpyrrolidone) of 16.9.

### Alternative Production Method for Polymer 1:

| | | |
|---|---|---|
| Initial | 180 g (0.02 mol) | ethoxylated/propoxylated polydimethyl- |
| charge | | siloxane, Mₙ = 9000 g/mol |
| | | (Pluriol ® ST 4005, BASF Aktiengesell- |
| | | schaft) |
| | 30.0 g | Acetone |
| Feed 1 | 27.7 g (0.125 mol) | Isophorone diisocyanate |
| Feed 2 | 20.0 g | Acetone |
| Feed 3 | 9.2 g (0.135 mol) | Imidazole |
| Feed 4 | 210 g | Ethanol |

In a 4-necked flask which was equipped with stirrer, dropping funnel, thermometer, reflux condenser and a device for working under nitrogen, the initial charge was heated to an internal temperature of 60° C. with stirring. Feed 1 was then added over the course of 10 min. The resulting reaction mixture was stirred for 3 h at an internal temperature of 85° C. The mixture was then left to cool to 30° C., and feed 2 was added and homogenized. This gave a polyurethane prepolymer with an NCO content of about 3%. At an internal temperature of 20° C., feed 3 was metered in over the course of 10 min and the resulting reaction mixture was then stirred for a further 20 min at a temperature of 40° C. The acetone is then removed under a gentle vacuum at an external temperature of 100° C. The polymer mass obtained in this way was diluted by adding feed 4 at a temperature of 40° C. This gave a pale yellow clear viscous solution with a solids content of about 50%. The product had a K value (measured on a 1% strength solution in N-methylpyrrolidone) of 17.0.

The urethane compounds 2 to 8 were produced analogously.

| | Pluriol ® | | | | | |
|---|---|---|---|---|---|---|
| | ST 4005 | PEG 300 | MDEA | Imidazole | DEG | IPDI |
| Example | (mol) | (mol) | (mol) | (mol) | (mol) | (m ol) |
| 1 | 0.02 | — | — | 0.135 | — | 0.125 |
| 2 | 0.02 | — | — | 0.10 | — | 0.10 |
| 3 | 0.02 | — | — | 0.08 | — | 0.80 |
| 4 | 0.02 | 0.02 | — | 0.12 | — | 0.14 |
| 5 | 0.02 | 0.02 | 0.02 | 0.12 | — | 0.15 |
| 6 | 0.02 | 0.02 | 0.02 | 0.12 | 0.01 | 0.16 |
| 7 | 0.02 | — | — | 0.13 | 0.02 | 0.15 |
| 8 | 0.02 | 0.02 | — | 0.11 | 0.02 | 0.15 |

Pluriol ® ST 4005 = ethoxylated/propoxylated polydimethylsiloxane, Mₙ = 900 g/mol
PEG 300 = polyethylene glycol M_{w} = 300 g/mol
MDEA = methyldiethanolamine
DEG = diethylene glycol
IPDI = isophorone diisocyanate

### II. Application Examples

### Example 1

| VOC 80 aerosol hairspray | [%] |
|---|---|
| Urethane compound from production example 1 | 0.20 |
| (50% strength solution in ethanol) | |
| Luvimer ® 100 P (powder) *) | 5.00 |
| Ethanol | 55.00 |
| Amino-2-methylpropanol (95% strength) | 1.80 |
| Propane/butane propellant gas | 38.00 |

Further additives: silicone, perfume, antifoam . . .
*) Luvimer ® 100 P: copolymer of t-butyl acrylate, ethyl acrylate and methacrylic acid (BASF Aktiengesellschaft)

The example can be repeated with the urethane compounds of production examples 2, 3, 4, 5, 6, 7 and 8. In each case, a VOC 80 aerosol hairspray with good properties is obtained.

In place of Luvimer® 100 P, the example can be repeated with all of the setting polymers commercially available, in particular with Luviset® P.U.R., Luviset® Si-PUR A, Luviskol® VA, Luviskol® Plus, Luviset® VBM, Luviset® CA66, Ultrahold Strong, Ultrahold 8, Luviflex® Silk, hair-setting agents under the name Amphomer®, such as Amphome® 28-4910, HC, LV 71, 30S, hair-setting agents under the name Balance®, such as Balance® 0/55, CR, 47, Acudyne® 258, hair-setting agents under the name Gantrez®, such as Gantrez® ES-225, ES-425, SP-215, H, M, hair-setting agents under the name Plascise®, such as Plascise® L-53P, L53D, L-8011C, hair-setting agents under the name Aquaflex®, such as Aquaflex® FX-64, SF40, Advantage® Plus, Amerhold® DR-25, Diahold® A-503, betaine hair-setting agents under the names Diaformer®, such as Diaformer® Z400 or Yukaformer®, such as Yukaformer® M-75, Aphoset®, hair-setting agents under the name Stepanhold®, such as Stepanhold® R-1, Extra, Mexomers®, vinyl acetate copolymers under the name Resyn®, such as Resyn® 28-2930, 28-1310, 28-3307.

In each case, a VOC 80 aerosol hairspray with good properties is obtained.

### Example 2

| VOC 80 hairspray | [%] |
|---|---|
| Luvimer ® 100 P (powder) | 5.00 |
| Ethanol | 40.00 |
| Water | 15.00 |
| Urethane compound 1 (50% in ethanol) | 0.20 |
| Amino-2-methylpropanol (95% strength) | 1.80 |
| Dimethyl ether propellant gas | 38.00 |

Further additives: silicone, perfume, antifoam . . .

The example can be repeated with the urethane compounds of production examples 2, 3, 4, 5, 6, 7 and 8. In each case, a VOC 80 aerosol hairspray with good properties is obtained.

In place of Luvimer® 100 P, the example can be repeated with all setting polymers which are commercially available, in particular with Luviset® P.U.R., Luviset® Si-PUR A, Luviskol® VA, Luviskol® Plus, Luviset® VBM, Luviset® CA66, Ultrahold Strong, Ultrahold 8, Luviflex® Silk, hair-setting agents under the name Amphomer®, such as Amphome® 28-4910, HC, LV 71, 30S, hair-setting agents under the name Balance®, such as Balance® 0/55, CR, 47, Acudyne® 258, hair-setting agents under the name Gantrez®, such as Gantrez® ES-225, ES-425, SP-215, H, M, hair-setting agents under the name Plascise®, such as Plascise® L-53P, L53D, L-8011C, hair-setting agents under the name Aquaflex® such as Aquaflex® FX-64, SF-40, Advantage® Plus, Amerhold® DR-25, Diahold® A-503, betaine hair-setting agents under the names Diaformer®, such as Diaformer® Z400, or Yukaformer®, such as Yukaformer® M-75, Aphoset®, hair-setting agents under the name Stepanhold®, such as Stepanhold® R-1, Extra, Mexomers®, vinyl acetate copolymers under the name Resyn®, such as Resyn® 28-2930, 28-1310, 28-3307.

In each case, a VOC 80 aerosol hairspray with good properties is obtained.

### Example 3

| VOC 55 hairspray | [%] |
|---|---|
| Luviset ® P.U.R. (30% water-ethanol) | 15.00 |
| Ethanol | 15.40 |
| Water | 31.40 |
| Urethane compound 1 (50% in ethanol) | 0.20 |
| adjust to pH 8.3 with amino-2-methylpropanol | |

Further additives: silicone, perfume, antifoam . . .

The example can be repeated with the urethane compounds of production examples 2, 3, 4, 5, 6, 7 and 8. In each case, a VOC 55 aerosol hairspray with good properties is obtained.

In place of Luviset® P.U.R., the example can be repeated with all VOC 55 setting polymers which are commercially available, in particular with Luviset® Si-PUR A, Luviskol® Plus, LuvisetO VBM, BalanceO 0/55, Balance® 47, Luvimer® Pro 55, Eastman® AQ 48, Stepanhold® R-1, Resyn® 28-2930. In each case, a VOC 55 aerosol hairspray with good properties is obtained.

### Example 4

| VOC 55 pump spray | [%] |
|---|---|
| Luviset ® P.U.R. (30% water-ethanol) | 15.00 |
| Ethanol | 53.40 |
| Water | 31.40 |
| Urethane compound 1 (50% in ethanol) | 0.20 |
| adjust to pH 8.3 with amino-2-methylpropanol | |

Further additives: silicone, perfume, antifoam . . .

The example can be repeated with the urethane compounds of production examples 2, 3, 4, 5, 6, 7 and 8. In each case, a VOC 55 Pump spray with good properties is obtained.

In place of Luviset® P.U.R., the example can be repeated with all VOC 55 setting polymers which are commercially available, in particular with Luviset® Si-PUR A, Luviskol® Plus, Luviset® VBM, Balance® 0/55, BalanceD 47, Luvimer® Pro 55, Eastman® AQ 48, Stepanhold® R-1, Resyn® 28-2930. In each case, a VOC 55 pump spray with good properties is obtained.

### Example 5

| VOC 20 pump spray | [%] |
|---|---|
| Luviset ® P.U.R. (30% water-ethanol) | 15.00 |
| Ethanol | 18.40 |
| Water | 66.40 |
| Urethane compound 1 (50% in ethanol) | 0.20 |
| adjust to pH 8.3 with amino-2-methylpropanol | |

Further additives: silicone, perfume, antifoam . . .

The example can be repeated with the urethane compounds of production examples 2, 3, 4, 5, 6, 7 and 8. In each case, a VOC 20 pump spray with good properties is obtained.

In place of Luviset® P.U.R., the example can be repeated with all VOC 55 setting polymers which are commercially available, in particular with Luviset® Si-PUR A, Luviskol® Plus, Luviset® VBM, Balance® 0/55, Balance® 47, Luvimer® Pro 55, Eastman® AQ 48, Stepanhold® R-1, Resyn® 28-2930.

In each case, a VOC 20 pump spray with good properties is obtained.

### Example 6

| VOC < 10 pump spray | [%] |
|---|---|
| Luviset ® Clear (20% strength) | 5.00 |
| Luviset ® P.U.R. (30% water-ethanol) | 10.00 |
| Urethane compound 1 (50% in ethanol) | 0.20 |
| Water | 84.80 |
| adjust to pH 8.3 with amino-2-methylpropanol | |

Further additives: silicone, perfume, antifoam . . .

The example can be repeated with the urethane compounds of production examples 2, 3, 4, 5, 6, 7 and 8. In each case, a VOC<10 pump spray with good properties is obtained.

In place of Luviset® P.U.R., the example can be repeated with all VOC 55 setting polymers commercially available, in particular with Luviset® Si-PUR A, Luviskol® Plus, Luviset® VBM, Balance® 0/55, Balance® 47, Luvimer® Pro 55, Eastman® AQ 48, Stepanhold® R-1, Resyn® 28-2930.

In each case, a VOC<10 pump spray with good properties is obtained.

### Example 7

| Foam conditioner | [%] |
|---|---|
| Urethane compound from Example No. 1 | 20.00 |
| (25% strength aqueous solution) | |
| Cremophor A 25 (Ceteareth 25/BASF) | 0.20 |
| Comperlan KD (Coamide DEA/Henkel) | 0.10 |
| Water | 69.70 |
| Propane/butane | 10.00 |

Further additives: perfume, preservative . . .

Production: weigh in and dissolve with stirring. Bottle and add propellant gas.

The example can be repeated with the urethane compounds of examples 2, 3, 4, 5, 6, 7 and 8. In each case, a foam conditioner with good properties is obtained.

### Example 8

| | |
|---|---|
| Hair gel containing Aculyn 28: | [%] |

Phase 1:

| | |
|---|---|
| Urethane compound from Example No. 1 | 12.00 |
| (25% strength aqueous solution) | |
| Water, dist. | 37.00 |
| Aminomethylpropanol (38% strength solution) | 1.00 |

Further additives: preservative, soluble ethoxylated silicone, perfume . . .

Phase 2:

| | |
|---|---|
| Aculyn 28 (1% strength aqueous suspension) | 50.00 |

### Production:

Phases 1 and 2 are weighed in separately and homogenized. Phase 2 is then slowly stirred into phase 1. An essentially clear, stable gel is formed.

The example can be repeated with the urethane compounds of examples 2, 3, 4, 5, 6, 7 and 8. In each case, a hair gel containing Aculyn 28 with good properties is obtained.

### Example 9

| | |
|---|---|
| Hair gel containing hydroxyethylcellulose: | [%] |

Phase 1:

| | |
|---|---|
| Urethane compound from Example No. 1 | 12.00 |
| (25% strength solution) | |
| Water, dist. | 30.00 |

Further additives: preservative, soluble ethoxylated silicone, perfume . . .

Phase 2:

| | |
|---|---|
| Natrosol HR 250 (5% strength solution) | 50.00 |
| | Hydroxyethylcellulose |
| | (Hercules) |

### Production:

Phases 1 and 2 are weighed in separately and homogenized. Phase 2 is then slowly stirred into phase 1. An essentially clear, stable gel is formed.

The example can be repeated with the urethane compounds of examples 2, 3, 4, 5, 6, 7 and 8. In each case, a hair gel containing hydroxyethylcellulose with good properties is obtained.

### Example 10

| | |
|---|---|
| Conditioner shampoo: | [%] |

| | | |
|---|---|---|
| A) | Texapon NSO 28% strength | 50.00 |
| | (sodium laureth sulfate/Henkel) | |
| | Comperlan KS (Coamide DEA/Henkel) | 1.00 |
| | Urethane compound from Example No. 1 | 20.00 |
| | (25% strength aqueous solution) | |
| | q.s. perfume oil | |
| B) | Water | 27.50 |
| | Sodium chloride | 1.50 |
| | q.s. preservative . . . | |

### Production:

Phases 1 and 2 are weighed in separately and homogenized. Phase 2 is then slowly stirred into phase 1. An essentially clear, stable gel is formed.

The example can be repeated with the urethane compounds of examples 2, 3, 4, 5, 6, 7 and 8. In each case, a conditioner shampoo with good properties is obtained.

### Example 11

Standard O/W cream:

| | |
|---|---|
| [%] | CTFA name |

Oil phase:

| | | |
|---|---|---|
| Cremophor A6 | 3.5 | Ceteareth-6 (and) stearyl |
| | | alcohol |
| Cremophor A25 | 3.5 | Ceteareth-25 |
| Glycerol monostearate s.e. | 2.5 | Glyceryl stearate |
| Paraffin oil | 7.5 | Paraffin Oil |
| Cetyl alcohol | 2.5 | Cetyl Alcohol |
| Luvitol EHO | 3.2 | Cetearyl Octanoate |
| Vitamin E acetate | 1.0 | Tocopheryl Acetate |
| Nip-Nip | 0.1 | Methyl and Propyl 4-hydroxy- |
| | | benzoate (7:3) |

Water phase:

| | | |
|---|---|---|
| Urethane compound from | 3.0 | |
| Example No. 1 | | |
| (25% strength solution) | | |
| Water | 74.6 | |
| 1,2-Propylene glycol | 1.5 | Propylene glycol |
| Germall II | 0.1 | Imidazolidinylurea |

### Production:

The oil and water phases are weighed in separately and homogenized at a temperature of about 80° C. The water phase is then slowly stirred into the oil phase and slowly cooled to room temperature with stirring.

The example can be repeated with the urethane compounds of examples 2, 3, 4, 5, 6, 7 and 8. In each case, a standard O/W cream with good properties is obtained.

### Example 12

### Body Lotion Foam

Phase A

| | |
|---|---|
| 1.50 | Ceteareth-25 |
| 1.50 | Ceteareth-6 |
| 4.00 | Cetearyl alcohol |
| 10.00 | Cetearyl octanoate |
| 1.00 | Dimethicone |

Phase B

| | |
|---|---|
| 3.00 | Urethane compound (20% strength aqueous solution) |
| 2.00 | Panthenol |
| 2.50 | Propylene glycol |
| q.s. | Preservative |
| 74.50 | dist. water |

Phase C

| | |
|---|---|
| q.s. | Perfume oil |

### Production:

Heat phases A and B separately to about 80° C. Stir phase B into phase A and homogenize. Cool to about 40° C., add phase C and briefly homogenize again. Bottling: 90% active ingredient and 10% propane/butane at 3.5 bar (20° C.).

The example can be repeated with the urethane compounds of examples 2, 3, 4, 5, 6, 7 and 8. In each case, a body lotion foam with good properties is obtained.

### Example 13

### Shower Gel

| | |
|---|---|
| 50.00 | Sodium laureth sulfate, magnesium laureth sulfate, |
| | sodium laureth-8 sulfate, magnesium laureth-8 |
| 1.00 | Cocoamide DEA |
| 4.00 | Urethane compound (25% strength aqueous solution) |
| 2.00 | Sodium laureth sulfate, glycol distearate, cocamide |
| | MEA, laureth-10 |
| q.s. | Preservative |
| q.s. | Perfume oil |
| 2.00 | Sodium chloride |
| 41.00 | dist. water |

### Production:

Weigh in all of the components together and stir until dissolved.

The example can be repeated with the urethane compounds of examples 2, 3, 4, 5, 6, 7 and 8. In each case, a shower gel with good properties is obtained.

### Example 14

### Shower Gel

| | |
|---|---|
| 30.00 | Sodium laureth sulfate |
| 6.00 | Sodium cocoamphodiacetate |
| 6.00 | Cocamidopropylbetaine |
| 3.00 | Sodium laureth sulfate, glycol distearate, cocamide MEA, |
| | laureth-10 |
| 7.70 | Polyquaternium-44 |
| 1.50 | Urethane compound (25% strength aqueous solution) |
| 1.00 | Panthenol |
| q.s. | Preservative |
| q.s. | Perfume oil |
| q.s. | Citric acid |
| 0.50 | Sodium chloride |
| 44.30 | dist. water |

### Production:

Weigh in the components of phase A and dissolve. Adjust the pH to 6 to 7.

The example can be repeated with the urethane compounds of examples 2, 3, 4, 5, 6, 7 and 8. In each case, a shower gel with good properties is obtained.

1. A urethane compound comprising
a) at least one polyether group-containing silicone derivative which comprises at least two groups which are reactive toward isocyanate groups,
b) at least one heterocycle with at least one ring nitrogen atom and at least one group which is reactive toward isocyanate groups and
c) at least one polyisocyanate.

2. The urethane compound according to claim 1, further comprising
d) at least one compound different from components a) and b) which comprises at least two groups which are reactive toward isocyanate groups.

3. The urethane compound according to claim 1, where component a) is chosen from:
polysiloxanes of the general formula I.1 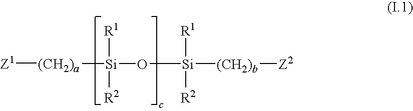
in which
a and b, independently of one another, are 1 to 8,
c is 2 to 1000,
R¹ and R², independently of one another, are alkyl, cycloalkyl or aryl,
Z¹ and Z², independently of one another, are radicals of the formula II
—(OCH₂CH₂)ᵤ(OCH(CH₃)CH₂)ᵥ(O(CH₂)₄)_{w}—X¹—H (II)
where
in formula II the order of the alkylene oxide units is arbitrary,
u, v and w, independently of one another, are an integer from 0 to 500, where the sum of u, v and
w>0,
X¹ is O or NR³, in which R³ is hydrogen, alkyl, cycloalkyl or aryl;
polysiloxanes of the general formula I.2 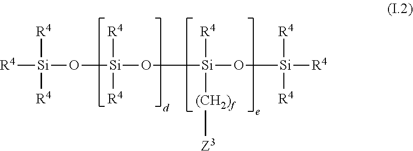
in which
the order of the siloxane units is arbitrary,
the radicals R⁴, independently of one another, are in each case alkyl, cycloalkyl or aryl,
d is an integer from 2 to 1000,
e is an integer from 2 to 100,
f is an integer from 2 to 8, and
Z³ is a radical of the formula II as defined above,
and mixtures thereof.

4. The urethane compound according to claim 1, where component b) is chosen from a nitrogen-containing heterocycle wherein the nitrogen-containing heterocycle is selected from the group consisting of purines, pyrazoles, imidazoles, triazoles, tetrazoles, piperazines, imidazolines, imidazolidines, pyrazolines, pyrazolidines and mixtures thereof.

5. The urethane compound according to claim 4, where component b) comprises imidazole or a derivative thereof or consists of imidazole or an imidazole derivative.

6. The urethane compound according to claim 1, where component b) is chosen from a nitrogen-containing heterocycle wherein the nitrogen-containing heterocycle is selected from the group consisting of pyridines, pyridazines, pyrimidines, pyrazines, 1,3,5-triazines, 1,2,4-triazines, 1,2,3-triazines, tetrazines, pyrroles, quinolines, isoquinolines, cinnolines, quinazolines, quinoxalines, phenazines, acridines, indoles, isoindoles, carbazoles, pyrrolines and pyrrolidines and wherein the nitrogen-containing heterocycle has at least one group which is reactive toward isocyanate groups as substituents.

7. The urethane compound according to claim 1, where component c) comprises at least one diisocyanate with two isocyanate groups of varying reactivity.

8. The urethane compound according to claim 1, where component c) comprises isophorone diisocyanate.

9. The urethane compound according to claim 2, where component d) is chosen from
d1) compounds with a molecular weight in the range from 56 to 280 g/mol which comprise two groups which are reactive toward isocyanate groups per molecule,
d2) polymers with a number-average molecular weight of more than 280 which have two groups which are reactive toward isocyanate groups per molecule,
and mixtures thereof.

10. A method of producing a urethane compound which comprises wherein the urethane compound comprises
a) at least one polyether group-containing silicone derivative which comprises at least two groups which are reactive toward isocyanate groups,
b) at least one heterocycle with at least one ring nitrogen atom and at least one group which is reactive toward isocyanate groups
c) at least one polyisocyanate, and
d) optionally at least one compound which is different from components a) and b) and which comprises at least two groups which are reactive toward isocyanate groups,
in which
i) in a first stage the compounds a) and the polyisocyanates c) and, optionally at least some of the compounds d) are reacted to give an isocyanate group-containing prepolymer, and
ii) in a second stage the prepolymer present in i) is reacted with the compounds b) and, if optionally, the compounds d) which have not already been used in step i).

11. The method according to claim 10, where at least one of the components a) and, optional component d) has a hydroxyl group-containing compound in which
i) all of the hydroxyl group-containing compounds a) and d) and the polyisocyanates c) are reacted at a temperature in a range from 55 to 120° C. in the absence of a solvent or in an aprotically polar solvent to give an isocyanate group-containing prepolymer, and
ii) the prepolymer obtained in stage i) is reacted with the compounds b) and, if present optionally, the amine group-containing compounds d) at a temperature in a range from 0 to 60° C. in an alcohol or alcohol/water mixture as solvent.

12. The method according to claim 10, in which the reaction in stage i) is carried out without the use of a solvent and in stage ii) in the presence of a cosmetically acceptable solvent.

13. A cosmetic or pharmaceutical composition comprising
A) at least one urethane compound as defined in claim 1, and
B) at least one cosmetically or pharmaceutically acceptable carrier.

14. A composition according to claim 13, which additionally comprises at least one acid group-containing polymer S).

15. The composition according to claim 13, which additionally comprises at least one polymer which comprises N-vinylpyrrolidone and/or N-vinylcaprolactam in copolymerized form.

16. The composition according to claim 13, where component B) is selected from the group consisting of
i) water,
ii) water-miscible organic solvents, preferably C₂-C₄-alkanols, in particular ethanol
iii) oils, fats, waxes,
iv) esters of C₆-C₃₀-monocarboxylic acids with mono-, di- or trihydric alcohols which are different from iii),
v) saturated acyclic and cyclic hydrocarbons,
vi) fatty acids,
vii) fatty alcohols,
viii) propellant gases
and mixtures thereof.

17. The composition according to claim 13, comprising at least one additive which is different from components A), B) and S) and is chosen wherein the at least one additive is selected from the group consisting of cosmetically active ingredients, emulsifiers, surfactants, preservatives, perfume oils, thickeners, hair polymers, hair and skin conditioners, graft polymers, water-soluble or dispersible silicone-containing polymers, photoprotective agents, bleaches, gel formers, care agents, colorants, tints, tanning agents, dyes, pigments, consistency regulators, humectants, regreasing agents, collagen, protein hydrolyzates, lipids, antioxidants, antifoams, antistats, emollients and softeners.

18. The composition according to claim 13 in the form of a gel, foam, spray, mousse, ointment, cream, emulsion, suspension, lotion, milk or paste.

19. The use of a urethane compound as defined in claim 1 in skin-cleansing compositions, compositions for the care and protection of the skin, nail care compositions, preparations for decorative cosmetics and hair-treatment compositions.

20. The use according to claim 19 in hair-treatment compositions as conditioners.

21. The use according to claim 20, where the composition is in the form of a hair gel, shampoo, setting foam, hair tonic, hairspray or hair foam.

22. The use of a urethane compound as defined in claim 1 as auxiliary in pharmacy, preferably as or in (a) coating(s) for solid drug forms, as surface-active compound, as or in (an) adhesive(s) and as or in (a) coating(s) for the textile, paper, printing and leather industry.
